(19) United States
(12) Patent Application Publication
(10) Pub. No.: US 2026/0176569 A1
(43) Pub. Date: Jun. 25, 2026

(54) A VALVE SYSTEM FOR A MODULAR INCUBATOR SYSTEM

(71) Applicant: ESCO MEDICAL TECHNOLOGIES, UAB, Kaunas (LT)

(72) Inventors: Ricky Lindgaard NIELSEN, Egå (DK); Vytautas VIRBICKAS, Kaunas (LT); Mikkel Hjerrild MIKKELSEN, Vejle (DK)

(21) Appl. No.: 19/124,545

(22) PCT Filed: Jun. 22, 2023
(86) PCT No.: PCT/EP2023/066970

§ 371 Date: Apr. 25, 2025

(30) Foreign Application Priority Data
Nov. 7, 2022 (DK) PA 2022 01008

(51) Int. Cl. C12M 1/00; G06F 1/16

(52) U.S. Cl. CPC C12M 41/14; G06F 1/1632

(57) ABSTRACT

A valve system for a modular incubator system includes a first valve and a second valve, each including a valve body and a valve element. Each valve body includes a front end and a rear end and a throughgoing channel extending between said front end and said rear end. Each valve element is arranged in the throughgoing channel of the valve body in such a way that said valve element is displaceable in a displacement direction D between a first extreme position and a second extreme position in said throughgoing channel, wherein in said first extreme position, said valve element is displaced in a direction towards the front end of said valve body, and wherein in said second extreme position, said valve element is displaced in a direction towards the rear end of said valve body.

### FIELD OF THE INVENTION

The present invention relates in general to the field of incubation of viable biological materials and in particular to incubators for IVF (in vitro fertilization) procedures.

More specifically, the present invention relates in a first aspect to a valve system for a modular incubator system.

In a second aspect the present invention relates to a modular incubator system for incubating a viable biological material.

In a third aspect the present invention relates to a modular incubator chamber for incubating a viable biological material.

In a fourth aspect the present invention relates to a docking station for docking a modular incubator chamber.

In a fifth aspect the present invention relates to a use of a valve system according to the first aspect in a modular incubator system.

In a sixth aspect the present invention relates to a use of a modular incubator system according to the second aspect for incubating a viable biological material.

In a seventh the present invention relates to a use of a modular incubator chamber according to the third aspect for incubating a viable biological material.

In an eight aspect the present invention relates to a use of a docking station according to the fourth aspect for incubating a viable biological material.

In a ninth aspect the present invention relates to a method of incubating a viable biological material.

### BACKGROUND OF THE INVENTION

The development of in vitro fertilization (IVF) has for the latest few decades resulted in considerably improved methods and techniques which have enhanced success rates of IVF mediated pregnancies and births.

In vitro fertilization involves capturing a ripened egg from a female ovary, fertilizing the ovary with a spermatozoon, incubating the fertilized egg under a controlled environment and subsequently inserting the fertilized and incubated egg in a female's uterus.

As in vitro fertilization is most commonly used by females or couples which notoriously are having problems in getting pregnant the natural way, thus implying some degree of reduced fertility by the male or female counterpart of the couple, or both, and as in vitro fertilization techniques involves quite expensive procedures, these in vitro fertilization techniques are usually performed in a way that seek to optimize efficiency, especially in view of the fact that frequently more than one insertion of a fertilized egg into the female' uterus will be necessary in order to encounter a successful pregnancy.

Additionally, compared to the natural way of getting pregnant, in respect of a couple wherein an individual is having a generic disease or in case of suspicion of such disease, an IVF mediated pregnancy may be advantageous.

Accordingly, in order to make the in vitro fertilization techniques efficient, the female is typically subjected to a hormone treatment prior to harvesting eggs from her ovary. Such hormone treatment will make the female ovary ovulate not only one egg, but a multitude of eggs at the same time.

In order to increase the chance of a viable and successful pregnancy more than one egg from the same female will accordingly be fertilized and incubated concurrently in an incubator.

Prior art incubators include a compartment which allows for accommodating more than one culture dish comprising the fertilized eggs.

Performing a successful in vitro fertilization and incubation of a fertilized egg is not an easy task. One of the major reasons for the rather low success rate of in vitro fertilizations is the absence of reliable methods for providing and maintaining optimum incubation conditions for the embryo.

Some improved prior art incubators comprise a housing having one or more doors for providing access to the interior of the incubator. The interior of the incubator holds one or more culture dishes accommodating the embryos to be cultured. Such incubators may be provided with various regulation means for controlling humidity, temperature and gas composition of the interior of the incubator.

Recently, smaller modular incubators have been introduced in the marked. These modular incubators are configured to be stored in docking ports in a docking station which may provide the controlling of physical and chemical parameters encountered by the embryos being accommodated therein. Once any manual manipulation steps in respect of the embryo is needed, such as manual inspection or adding or removing or exchanging growth media, the modular incubator may be removed from the docking station and arranged on a laboratory bench for easy access to the embryo.

These modular incubators and the corresponding docking ports of the docking station may even be provided with gas connectors so that once a modular incubator is docked in a docking port of the docking station, gas connectors of the modular incubator and corresponding gas connectors of the docking port engage in such a way that a gas having a desired gas composition may be conveyed from the docking station to each modular incubator via these gas connectors. In this way a desired gas composition can be upheld in the interior of the modular incubators in the period of time where a modular incubator is being docked in the docking port of the docking station.

The gas composition is typically controlled by use of a gas mixing box enabling addition of CO₂ and N₂. An outlet of the gas mixing box is fluidly connected to an inlet gas connector of each modular incubator and a return inlet of the gas mixing box is fluidly connected to an outlet gas connector of each modular incubator. Accordingly, gas is circulated through the modular incubator and through the gas mixing box. Upon controlling the gas composition to be supplied to the incubators, CO₂ is added to the gas mixing box in order to reach a desired CO₂ concentration and N₂ is being added to the gas mixing box in order to lower the O₂ level to a desired concentration. As leaks in the gas supply system is inevitably, small amounts of atmospheric air will find its way into the system and therefore oxygen will never be depleted below the desired oxygen concentration.

By constantly monitoring the CO₂ concentration and the O₂ concentration of the gas circulating in the system and by constantly regulating the gas composition leaving the gas mixing box, in response to any deviations from the desired and predetermined gas composition, it possible to ensure that the gas leaving the gas mixing box to be supplied to the modular incubators is having the desired and predetermined optimum composition.

However, once a modular incubator is removed from the docking station for manual inspection or for performing other processing steps, no gas supply into the modular incubator having a desired composition is being provided. Moreover, in such a situation, ambient air may mix with the desired gas composition which is located in the interior of the modular incubator, thereby ultimately leading to a gas composition in the interior of the incubator chamber which to a large extent deviates from an optimum composition as stipulated by preferred and predetermined incubation protocols.

Additionally, when removing a modular incubator from the docking station, ambient air may find its way into the gas distribution system of the docking station which provides for circulating gas from the gas source, therefrom to the docking ports, further into and through the modular incubator which is being docked in the docking station and finally back to the gas source.

In this way, ambient air entering the interior of the modular incubator and/or entering into the gas distribution system upon removal of a modular incubator from its docking port may imply contamination with such ambient air not only in the modular incubator removed, but also in the gas distribution system supplying gas to the remainder of the modular incubators. In this way, gas having a non-optimum gas composition will be circulated in the gas distribution system and be supplied to the modular incubators.

It has been found that in respect of gas composition in the interior of an incubator chamber, even small deviations from what is considered to be an optimum gas composition, may have detrimental effects on the quality of the biological material being incubated therein.

Such detrimental effects may accordingly represent enhanced risks that the IVF procedure ends in an unsuccessful pregnancy once the embryo has been inserted into a female's uterus.

Accordingly, a need persists for improved modular incubators.

It is an objective of the present invention to fulfill such need.

### BRIEF DESCRIPTION OF THE INVENTION

These objectives are fulfilled according to the various aspect of the present invention.

Accordingly, the first aspect of the present invention relates to a valve system for a modular incubator system, wherein said valve system comprises a first valve and a second valve;
wherein said first valve comprises:
   a first valve body; and
   a first valve element;
wherein said first valve body comprising a front end and a rear end;
wherein said first valve body comprises a first throughgoing channel extending between said front end and said rear end of said first valve body;
wherein said first valve element is being arranged in said first throughgoing channel of said first valve body in such a way that said first valve element is being displaceable in a displacement direction D between a first extreme position and a second extreme position in said first throughgoing channel, wherein in said first extreme position, said first valve element is being displaced in a direction towards the front end of said first valve body, and wherein in said second extreme position, said first valve element is being displaced in a direction towards the rear end of said first valve body;
wherein the dimensions and geometries of said first valve body and said first valve element are adapted to each other in such a way that, once being positioned in said first extreme position, said first valve element will block passage through said first throughgoing channel between said front end and said rear end thereof, and in such a way, that once being displaced in a direction towards said second extreme position, said first valve element will provide passage through said first throughgoing channel between said front end and said rear end thereof;
wherein said second valve comprises:
   a second valve body; and
   a second valve element;
wherein said second valve body comprising a front end and a rear end;
wherein said second valve body comprises a second throughgoing channel extending between said front end and said rear end of said second valve body;
wherein said second valve element is being arranged in said second throughgoing channel of said second valve body in such a way that said second valve element is being displaceable in a displacement direction D between a first extreme position and a second extreme position in said second throughgoing channel, wherein in said first extreme position, said second valve element is being displaced in a direction towards the front end of said second valve body, and wherein in said second extreme position, said second valve element is being displaced in a direction towards the rear end of said second valve body;
wherein the dimensions and geometries of said second valve body and said second valve element are adapted to each other in such a way that, once being positioned in said first extreme position, said second valve element will block passage through said second throughgoing channel between said front end and said rear end thereof, and in such a way, that once being displaced in a direction towards said second extreme position, said second valve element will provide passage through said second throughgoing channel between said front end and said rear end thereof.

The second aspect of the present invention relates to a modular incubator system for incubating a viable biological material M, said modular incubator system comprising:
one or more modular incubator chambers in combination with
a docking station;
wherein in respect of one or more of said one or more modular incubator chambers, said modular incubator chamber comprises a housing having a first end and a second end, thereby defining a longitudinal direction X between said first end and said second end;
wherein said housing comprises a lid, wherein said lid is being configured to be able to shift between an open configuration allowing access to the interior of said modular incubator chamber and a closed configuration, sealing off access to the interior of said modular incubator chamber;
wherein said modular incubator chamber, at said interior thereof, comprises a culture dish support for positioning a culture dish with the view to accommodate one or more biological materials M within the housing of said modular incubator chamber;
wherein in respect of one or more of said one or more modular incubator chambers, said housing of said modular incubator chamber comprises a chamber inlet opening for gas, wherein said chamber inlet opening for gas is being in fluid connection with the interior of said modular incubator chamber; and
wherein said housing of said modular incubator chamber furthermore comprises a chamber outlet opening for gas, wherein said chamber outlet opening for gas is being in fluid connection with the interior of said modular incubator chamber;
wherein said docking station comprises one or more docking ports for receiving a modular incubator chamber;
wherein in respect of one or more docking ports of said docking station, said docking port comprises a docking port outlet opening for gas; thereby enabling transfer of gas from said docking port of said docking station to the interior of said modular incubator chamber via said docking port outlet opening for gas and said chamber inlet opening for gas; and
wherein said docking port furthermore comprises a docking port inlet opening for gas, thereby enabling transfer of gas from the interior of said modular incubator chamber to said docking port of said docking station;
wherein one valve of the valve system of the first aspect of the present invention is being arranged in said chamber inlet opening for gas, and wherein another valve of the valve system of the first aspect of the present invention is being arranged in said docking port outlet opening for gas; and
wherein one valve of the valve system of the first aspect of the present invention is being arranged in said chamber outlet opening for gas, and wherein another valve of the valve system of the first aspect of the present invention is being arranged in said docking port inlet opening for gas.

The third aspect of the present invention relates to a modular incubator chamber, wherein said modular incubator chamber comprises a housing having a first end and a second end, thereby defining a longitudinal direction X between said first end and said second end;
wherein said housing comprises a lid, wherein said lid is being configured to be able to shift between an open configuration allowing access to the interior of said modular incubator chamber and a closed configuration, sealing off access to the interior of said modular incubator chamber;
wherein said modular incubator chamber, at said interior thereof, comprises a culture dish support for positioning a culture dish with the view to accommodate one or more biological materials M within the housing of said modular incubator chamber;
wherein said housing of said modular incubator chamber comprises a chamber inlet opening for gas, wherein said chamber inlet opening for gas is being in fluid connection with the interior of said modular incubator chamber; wherein one valve of the valve system according to the first aspect of the present invention is being arranged in said chamber inlet opening for gas;
wherein said housing of said modular incubator chamber furthermore comprises a chamber outlet opening for gas, wherein said chamber outlet opening for gas is being in fluid connection with the interior of said modular incubator chamber; wherein one valve of the valve system according to the first aspect of the present invention is being arranged in said chamber outlet opening for gas.

The fourth aspect of the present invention relates to a docking station, wherein said docking station comprises one or more docking ports for receiving a modular incubator chamber;
wherein in respect of one or more docking ports of said docking station, said docking port comprises a docking port outlet opening for gas; thereby enabling transfer of gas from said docking port of said docking station to an interior of said modular incubator chamber via said docking port outlet opening for gas; wherein one valve of the valve system according to the first aspect of the present invention is being arranged in said docking port outlet opening for gas; and
wherein said docking port furthermore comprises a docking port inlet opening for gas, thereby enabling transfer of gas from the interior of a modular incubator chamber to said docking port of said docking station; wherein one valve of the valve system according to the first aspect of the present invention is being arranged in said docking port inlet opening for gas.

In a fifth aspect the present invention provides a use of a valve system according to the first aspect of the present invention in a modular incubator system.

In a sixth aspect the present invention provides a use of a modular incubator system according to the second aspect of the present invention for incubating a viable biological material.

In a seventh aspect the present invention provides a use of a modular incubator chamber according to the third aspect of the present invention for incubating a viable biological material.

In an eighth aspect the present invention provides a use of a docking station according to the fourth aspect of the present invention for incubating a viable biological material.

In a ninth aspect the present invention provides a method of incubating a viable biological material.

The present invention in its various aspects allows upholding an optimum gas composition in a modular incubator chamber which is configured to be docked in a docking port of a docking station, even in situations where such a modular incubator chamber is being undocked from its associated docking port of the docking station.

Moreover, the present invention in its various aspects ensures that gas is not leaking out of inlet openings and outlet openings of a docking port of the docking station of the modular incubator system, when a modular incubator chamber is not being docking in that docking port.

### BRIEF DESCRIPTION OF THE FIGURES

FIG. 1 is a cut-through cross-sectional view of the valve system of the first aspect of the invention in which the two valves are separated from each other.

FIG. 2 is a cut-through cross-sectional view of the valve system of FIG. 1 in which the two valves are shown in a situation of having approached each other.

FIG. 3 is a cut-through cross-sectional view of the valve system of FIG. 1 in which the two valves are shown in a situation so close to each other that they start to open.

FIG. 4 is a cut-through cross-sectional view of the valve system of FIG. 1 in which the two valves have been fully engaged so that they are both open.

FIG. 5 is a perspective view illustrating a modular incubator system according to the second aspect of the present invention.

FIG. 6 is a perspective view showing a modular incubator chamber of the docking system of the second aspect of the present invention.

FIG. 7 is a top view of the modular incubator chamber illustrated in FIG. 6.

FIG. 8 is a plan rear view of the modular incubator chamber illustrated in FIGS. 6 and 7.

FIG. 9 is a cross-sectional view of the modular incubator chamber illustrated in FIGS. 6, 7 and 8.

FIG. 10 is a diagram illustrating one embodiment of a design of a gas supply system comprising a gas source and a gas distribution system to be used with the docking station of the modular incubator system of the present invention.

FIG. 11 is a diagram illustrating the concept of the gas supply system which in incorporated in the docking station of the modular incubator system of the present invention.

FIG. 12 is a diagram illustrating the working mode of the controlling of the modular incubator system according to the invention.

### DETAILED DESCRIPTION OF THE INVENTION

### The First Aspect of the Present Invention

In a first aspect the present invention relates to a valve system 100 for a modular incubator system 500, wherein said valve system comprises a first valve 2 and a second valve 4;
wherein said first valve 2 comprises:
   a first valve body 6; and
   a first valve element 8;
wherein said first valve body 6 comprising a front end 10 and a rear end 12;
wherein said first valve body 6 comprises a first throughgoing channel 14 extending between said front end 10 and said rear end 12 of said first valve body 6;
wherein said first valve element 8 is being arranged in said first throughgoing channel 14 of said first valve body 6 in such a way that said first valve element 8 is being displaceable in a displacement direction D between a first extreme position and a second extreme position in said first throughgoing channel 14, wherein in said first extreme position, said first valve element 8 is being displaced in a direction towards the front end 10 of said first valve body 6, and wherein in said second extreme position, said first valve element 8 is being displaced in a direction towards the rear end 12 of said first valve body 6;
wherein the dimensions and geometries of said first valve body 6 and said first valve element 8 are adapted to each other in such a way that, once being positioned in said first extreme position, said first valve element 8 will block passage through said first throughgoing channel 14 between said front end 10 and said rear end 12 thereof, and in such a way, that once being displaced in a direction towards said second extreme position, said first valve element 8 will provide passage through said first throughgoing channel 14 between said front end 10 and said rear end 12 thereof;
wherein said second valve 4 comprises:
   a second valve body 16; and
   a second valve element 18;
wherein said second valve body 16 comprising a front end 20 and a rear end 22;
wherein said second valve body 16 comprises a second throughgoing channel 24 extending between said front end 20 and said rear end 22 of said second valve body 16;
wherein said second valve element 18 is being arranged in said second throughgoing channel 24 of said second valve body 16 in such a way that said second valve element 18 is being displaceable in a displacement direction D between a first extreme position and a second extreme position in said second throughgoing channel 24, wherein in said first extreme position, said second valve element 18 is being displaced in a direction towards the front end 20 of said second valve body 16, and wherein in said second extreme position, said second valve element 18 is being displaced in a direction towards the rear end 22 of said second valve body 16;
wherein the dimensions and geometries of said second valve body 16 and said second valve element 18 are adapted to each other in such a way that, once being positioned in said first extreme position, said second valve element 18 will block passage through said second throughgoing channel 24 between said front end 20 and said rear end 22 thereof, and in such a way, that once being displaced in a direction towards said second extreme position, said second valve element 18 will provide passage through said second throughgoing channel 24 between said front end 20 and said rear end 22 thereof.

The valve system 100 of the first aspect of the present invention is intended for being used in a modular incubator system 500 in combination with a plurality of modular incubator chambers 300 as further described below. The modular incubator system 500 comprises a docking station 400 which comprises a plurality of docking ports 402 and the modular incubator chambers 300 are configured to be docked in a docking port 402 of the docking station 402.

By providing the docking ports 402 with an inlet and an outlet valve 4,2 and by providing each of the modular incubator chambers 300 with an inlet and an outlet valve 2,4 it is possible to provide gas having a predetermined and desired gas composition into and out of the modular incubator chambers 300, and only to allow passage through the valves 2,4 of the valve system of the modular incubator system 500 when a modular incubator chambers 300 is being docked in a docking port 402 of the docking station 400 of the modular incubator system 500. In this way a desired gas composition can be upheld in the interior of the modular incubator chambers 300, whether or not the modular incubator chamber 300 is being docked in a docking port or not. Moreover, the valves 4,2 in the docking ports prevents that or at least considerably reduces the amount of atmospheric air which may enter into the gas distribution system 204 which provides gas, by circulation, to the docking ports 402 of the docking system of the modular incubator system 500.

Hereby the magnitude of deviation from an optimum and desired and predetermined gas composition of gas flowing in that gas distribution system 204 will be reduced which ultimately contributes to enabling incubation of a viable biological material at optimum incubation conditions.

In an embodiment of the valve system according to the first aspect of the present invention, the dimensions and geometries of said first valve element 8 and said second valve element 18 are being adapted to each other in such a way that upon bringing said first valve 2 into contact with said second valve 4, by making their respective front ends 10,20 approach each other, said second valve element 18 of said second valve 4 is configured to displace said first valve element 8 of said first valve 2 towards the second extreme position thereof, thereby opening said first valve 2, and further, said first valve element 8 of said first valve 2 is configured to displace said second valve element 18 of said second valve 4 towards the second extreme position thereof, thereby opening said second valve 4.

Accordingly, in this way each the two valves 2,4 of the valve system 100 will make the other valve attain an open configuration, once being brought into contact.

In an embodiment of the valve system according to the first aspect of the present invention the first valve 2 comprises a first spring 26, wherein said first spring is adapted to interact with said first valve element 8, relative to said first valve body 6, in such a way that said first spring 26 will displace said first valve element 8, when not otherwise acted upon, towards said first extreme position thereof, thereby closing said first valve 2; and/or wherein said second valve 4 comprises a second spring 28, wherein said second spring is adapted to interact with said second valve element 18, relative to said second valve body 16, in such a way that said second spring 28 will displace said second valve element 18, when not otherwise acted upon, towards said first extreme position thereof, thereby closing said second valve 4.

The springs 26,28 will make the two valves 2,4 of the valve system 100 attain closed configuration when not in contact.

In an embodiment of the valve system according to the first aspect of the present invention the first valve 2 comprises said first spring 26 having first spring constant and wherein said second valve 4 comprises said second spring 28 having a second spring constant, wherein said first spring constant is equal to said second spring constant, thereby making said first valve 2 and said second valve 4 open approximately simultaneous upon being brought into contact with each other, or wherein said first spring constant is smaller than said second spring constant, thereby making said first valve 2 open before said second valve 4, upon being brought into contact with each other; or wherein said first spring constant is larger than said second spring constant, thereby making said second valve 4 open before said first valve 2, upon being brought into contact with each other.

In an embodiment of the valve system according to the first aspect of the present invention the first throughgoing channel 14 of said first valve 2 comprises a widened portion 30 having a first wall segment 32 defining a first inclined surface portion 34 which is being inclined relative to the direction of displacement D of said first valve element 8, and wherein said first valve element 8 comprises a widened portion 36 having a first contact surface 38, wherein said widened portion 36 of said first valve element 8 is being accommodated in said widened portion 30 of said first through-going channel 14 in such a way that when said first valve element 8 is being in its first extreme position, said first contact surface 38 of said first valve element 8 is being in contact with said first inclined surface portion 34 of said first throughgoing channel 14, thereby rendering said first valve 2 closed by blocking passage through said first through-going channel 14; and in such a way that when said first valve element 8 is being in its second extreme position, said first contact surface 38 of said first valve element 8 is being separated from said first inclined surface portion 34 of said first throughgoing channel 14, thereby rendering said first valve 2 open by providing passage through said first through-going channel 14.

In an embodiment of the valve system according to the first aspect of the present invention the first contact surface 38 of said first valve element 8 is being inclined relative to the direction of displacement D of said first valve element 8.

Accordingly, in this way the distance between first contact surface 38 of the valve element 6 and the first inclined surface portion 34 of the through-going channel 14 will determine whether or not the valve 2 will be open or closed.

In an embodiment of the valve system according to the first aspect of the present invention the first inclined surface portion 34 of said first throughgoing channel 14 and/or said first contact surface 38 of said first valve element 8 is/are having an inclination, relative to the direction D of displacement of said first valve element 8, of 5-90°, such as 10-85°, for example 15-80°, e.g. 20-75°, such as 25-70°, such as 30-65°, for example 35-60°, e.g. 40-55° or 45-50°.

These magnitudes of inclination will enable the opening/closing functionality of the valve 2.

In an embodiment of the valve system according to the first aspect of the present invention a first valve gasket 40 is provided in the area of said first contact surface 38 of said first valve element 8.

In one embodiment the first valve gasket 40 is being part of said first inclined surface portion 34 of said first throughgoing channel 14; and/or wherein said first valve gasket 40 is being part of said first contact surface 38 of said first valve element 8.

The gasket 26 will improve the air-tight nature of the valve 2 in its closed configuration.

In an embodiment of the valve system according to the first aspect of the present invention the second throughgoing channel 24 of said second valve 4 comprises a widened portion 42 having a second wall segment 44 defining a second inclined surface portion 46 which is being inclined relative to the direction of displacement D of said second valve element 18, and wherein said second valve element 18 comprises a widened portion 48 having a second contact surface 50, wherein said widened portion 48 of said second valve element 18 is being accommodated in said widened portion 42 of said second through-going channel 24 in such a way that when said second valve element 18 is being in its first extreme position, said second contact surface 50 of said second valve element 18 is being in contact with said second inclined surface portion 46 of said second throughgoing channel 24, thereby rendering said second valve 4 closed; and in such a way that when said second valve element 18 is being in its second extreme position, said second contact surface 50 of said second valve element 18 is being separated from said second inclined surface portion 46 of said second throughgoing channel 24, thereby rendering said first valve 4 open.

Accordingly, in this way the distance between second contact surface 50 of the valve element 16 and the second inclined surface portion 46 of the through-going channel 24 will determine whether or not the valve 4 will be open or closed.

In an embodiment of the valve system according to the first aspect of the present invention the second contact surface 50 of said second valve element 18 is being inclined relative to the direction of displacement D of said second valve element 18.

In an embodiment the second inclined surface portion 46 of said second throughgoing channel 24 and/or wherein said second contact surface 50 of said second valve element 18 is/are is having an inclination, relative to the direction of displacement of said second valve element 18 of 5-90°, such as 10-85°, for example 15-80°, e.g. 20-75°, such as 25-70°, such as 30-65°, for example 35-60°, e.g. 40-55° or 45-50°.

These magnitudes of inclination will enable the opening/closing functionality of the valve 4.

In an embodiment of the valve system according to the first aspect of the present invention the second valve gasket 52 is provided in the area of said second inclined surface portion 46 of said second throughgoing channel 24.

In an embodiment the second valve gasket 52 is being part of said second inclined surface portion 46 of said second throughgoing channel 24; and/or wherein said second valve gasket 52 is being part of said second contact surface 50 of said second valve element 18.

The gasket 28 will improve the air-tight nature of the valve 4 in its closed configuration.

In an embodiment the second valve gasket 52 comprises one or more lip portions 54, such as one or more tapered lip portions;
wherein said second valve gasket 52 is being part of said second inclined surface portion 46 of said second throughgoing channel 24 and wherein said one or more lip portions 54 is/are pointing towards said second contact surface 50 of said second valve element 18; or
wherein said second valve gasket 52 is being part of said second contact surface 50 of said second valve element 18 and wherein said one or more lip portions 54 is/are pointing towards second inclined surface portion 46 of said second throughgoing channel 24.

Such lip portions will improve the air-tight nature of the valve 4 in its closed configuration because of their resilient nature and because a relative high pressure on an outer side of the lip portion will make the lip portion press against the opposing surface of either the valve element 16 or of the second inclined surface portion 46 depending on the location of the second valve gasket 52, and thereby former a tighter seal.

In an embodiment of the valve system according to the first aspect of the present invention the valve body 6 of said first valve 2, at the front end 10 thereof, comprises a depression 56, and wherein the valve body 16 of said second valve 4, at the front end 20 thereof, comprises a hollow protrusion 58 surrounding at least part of said second valve element 18 of said second valve 4, wherein the dimensions and the geometry of said depression 56 and said protrusion 58 are adapted to each other in such a way that said protrusion 58 of said second valve body 16 will fit into said depression 56 of said first valve body 6.

Hereby leak of atmospheric air into the interior 306 of the modular incubator chamber 300 and/or into the inlet opening 404 or the outlet opening 406 of the docking port 402 will be reduced. Such leak of atmospheric air into the interior of the modular incubator chamber 306 and/or into the inlet opening 404 or the outlet opening 406 of the docking port 402 could imply deviations from the predetermined and desired gas composition to be supplied to the interior 306 modular incubator chambers 300.

In an embodiment of the valve system according to the first aspect of the present invention the first valve body 6 at the front end 10 thereof and at an inner end of said depression, comprises an end gasket 60, wherein said end gasket 60 surrounds at least part of said first valve element 8 and/or said first through-going channel 14 of said first valve 2, thereby allowing said protrusion 58 of said second valve body 16 of said second valve 4 to abut said end gasket 60, when said protrusion 58 of said second valve body 16 of said second valve 4 in being inserted into said depression 56 of said first valve body 6 of said first valve 2 in order to avoid leaking of gas.

Hereby the above-mentioned leaks will be further suppressed.

In an embodiment of the valve system according to the first aspect of the present invention the first valve element 8 comprises a first part 8a and a second part 8b, wherein said first part 8a of said first valve element 8 is being arranged proximate to said front end 10 of said first valve body 6, and wherein said second part 8b of said first valve element 8 is being arranged distal to said front end 10 of said first valve body 6; wherein said first part 8a and said second part 8b of said first valve element 8 are being connected to each other via a threaded tap/threaded hole arrangement 62.

Hereby adjustment of the total length of said first valve element 8, in a direction parallel to the direction D of displacement of said first valve element 8 is enabled, and thereby the degree of opening of the first valve 2 can be adjusted upon being approached to the other valve 4 of the valve system 100.

In an embodiment of the valve system according to the first aspect of the present invention the first valve element 8, at the end proximate to the front end 10 of said first valve body 6, comprises one or more through-going holes 64 for allowing conveying of gas through said holes into said first throughgoing channel 14 of said first valve body 6.

In an embodiment of the valve system according to the first aspect of the present invention the first gaskets, the second gasket and the end gasket independently is being made of a resilient polymer, such as rubber or silicone

### The Second Aspect of the Present Invention

In a second aspect the present invention relates to a modular incubator system 500 for incubating a viable biological material M, said modular incubator system comprising:
one or more modular incubator chambers 300 in combination with
a docking station 400;
wherein in respect of one or more of said one or more modular incubator chambers 300, said modular incubator chamber 300 comprises a housing 302 having a first end 340 and a second end 342, thereby defining a longitudinal direction X between said first end and said second end;
wherein said housing comprises a lid 304, wherein said lid is being configured to be able to shift between an open configuration allowing access to the interior 306 of said modular incubator chamber and a closed configuration, sealing off access to the interior of said modular incubator chamber;
wherein said modular incubator chamber 300, at said interior 306 thereof, comprises a culture dish support 308 for positioning a culture dish 310 with the view to accommodate one or more biological materials M within the housing 302 of said modular incubator chamber 300; wherein in respect of one or more of said one or more modular incubator chambers 300, said housing of said modular incubator chamber 300 comprises a chamber inlet opening for gas 312, wherein said chamber inlet opening for gas 312 is being in fluid connection with the interior 306 of said modular incubator chamber; and
wherein said housing 302 of said modular incubator chamber furthermore comprises a chamber outlet opening for gas 314, wherein said chamber outlet opening for gas 314 is being in fluid connection with the interior 306 of said modular incubator chamber;
wherein said docking station 400 comprises one or more docking ports 402 for receiving a modular incubator chamber;
wherein in respect of one or more docking ports 402 of said docking station 400, said docking port 402 comprises a docking port outlet opening for gas 404; thereby enabling transfer of gas from said docking port 402 of said docking station 400 to the interior 302 of said modular incubator chamber 300 via said docking port outlet opening for gas 404 and said chamber inlet opening for gas 312; and
wherein said docking port 402 furthermore comprises a docking port inlet opening for gas 406, thereby enabling transfer of gas from the interior 306 of said modular incubator chamber 300 to said docking port 402 of said docking station 400;
wherein one valve 2,4 of the valve system 100 of the first aspect of the present invention is being arranged in said chamber inlet opening for gas 312, and wherein another valve 4,2 of the valve system 100 of the first aspect of the present invention is being arranged in said docking port outlet opening for gas 404; and
wherein one valve 2,4 of the valve system 100 of the first aspect of the present invention is being arranged in said chamber outlet opening for gas 314, and wherein another valve 4,2 of the valve system 100 of the first aspect of the present invention is being arranged in said docking port inlet opening for gas 406.

In the present invention the term “modular incubator system” shall be construed to mean a system comprising a docking station in combination with one or more incubator chambers, wherein the one or more incubator chambers is/are configured to be docked in respective docking ports of that docking station. The modular incubator system is intended for incubation or cultivation of a viable biological material.

The incubator system comprising the docking station and one or more incubator chamber(s) in general is configured for providing some kind of interaction between the docking station and the incubator chambers being docked therein.

Such interactions may be one or more of the following: providing a gas having a desired composition to the incubator chamber(s); providing electricity to the incubator chamber(s) for powering hearing elements thereof and/or for powering a light source in the incubator chamber(s); allowing monitoring of the viable biological material being present in the incubator chamber(s), such as by means of an image capturing device which is located in the docking station.

In should be understood that within the meaning of the present application, the term “modular incubator system” shall be construed in such a way that the incubator chambers are configured to be used for incubation of a viable biological material, irrespective of whether the individual incubator chamber is being docked in a docking port of the docking station, or whether that incubator chamber is removed from the docking port of the docking station.

In this way, it is to be understood that cultivation or incubation of a viable biological material of the individual incubator chambers may take place and/or be continued even after that incubator chamber has been removed from its docking station and placed e.g. on a laboratory bench. Hereby manual manipulation operations, such as shift or control of culture or growth media, manual inspection by use of a laboratory microscope or the like can take place. Such operation are preferably carried out under a hood providing a desired gas atmosphere.

In preferred embodiment, and in order to make such manual manipulation operations practical conceivable, when the individual incubation chamber has been removed from a docking port, the incubation chamber is configured in a way that enables support on a planar, horizontal support surface. This may be attained by providing the bottom part of the incubator chamber with one or more supports or simply by making the bottom part of the incubator chamber comprise a flat surface.

In preferred embodiments the incubation chamber, in the orientation intended during use for incubation, is having its maximum dimension in a horizontal direction.

In this way the dimension of the incubation chamber in a horizontal direction is greater than the dimension in a vertical direction. Hereby, adequate stability is attended when the incubator chamber is used for incubation at a location outside a docking port of the docking station.

The individual incubator chambers may in embodiments comprise a display, such as an electronic display, for providing information relating to the identity of the viable biological material being accommodated in the incubator chamber.

It should be understood that in some embodiments the present invention does not relate to methods or uses which involve treatment of the human or animal body by surgery or diagnostic methods practiced on the human or animal body.

It should also be understood that that in other embodiments the present invention may relate to methods or uses which involve treatment of the human or animal body by surgery or diagnostic methods practiced on the human or animal body.

In an embodiment of the modular incubator system according to the second aspect of the present invention and in respect of one or more of said one or more modular incubator chambers 300, and in respect of one or more of said one or more docking ports 402 of said docking station 400, the position of said chamber inlet opening for gas 312 of said modular incubator chamber 300 and the position of said docking port outlet opening for gas 404 of said docking port 402 are adapted to each other in such a way that once docking said modular incubator chamber 300 in said docking port 402, said valve 2,4 of said chamber inlet opening for gas 312 of said housing 302 of said modular incubator chamber 300 and said valve 4,2 of said docking port outlet opening for gas 404 of said docking port 402 will be in fluid connection and in their open configuration; and in such a way that the position of said valve 4,2 of said chamber outlet opening for gas 314 of said modular incubator chamber 300 and the position of said valve 4,2 of said docking port inlet opening for gas 406 of said docking port 402 are adapted to each other in such a way that once docking said modular incubator chamber 300 in said docking port 402, said chamber outlet opening for gas 314 of said housing 302 of said modular incubator chamber 300 and said docking port inlet opening for gas 406 of said docking port 402 will be in fluid connection and in their open configuration.

Hereby merely docking a modular incubator chamber 300 into a docking port 402 of the docking station 400 of the modular incubator system 500 will imply automatically opening of the valves 2,4 of the valve system with the view to supply gas into and out of the interior 306 of the modular incubator chamber 300. Likewise, when a modular incubator chamber 300 is removed a docking port 402, the valves 2,4 of the modular incubator chamber 300 and the docking port 402 will automatically shut off passage of gas through these valves.

In an embodiment of the modular incubator system according to the second aspect of the present invention and in respect of one or more of said one or more modular incubator chambers 300, said housing 302 of said modular incubator chamber 300 comprises a transparent window 316, and wherein in respect of one or more docking ports 402 of said docking station 400, said docking port comprises an image capturing device 408, thereby allowing capturing images of a biological material M being accommodated in the interior 306 of a modular incubator chamber 300, once being docked in said docking port 402.

Hereby capturing images of a biological material M being accommodated in the interior 306 of a modular incubator chamber 300, once being docked in said docking port 402 in enabled.

In an embodiment of the modular incubator system according to the second aspect of the present invention and in respect of one or more of said one or more modular incubator chambers 300 and in respect of one or more of said one or more docking ports 402 of said docking station 400, the position of said transparent window 316 of said modular incubator chamber 300 is adapted to the position of said image capturing device 408 in said docking port 402 in a way that enables capturing of images by said image capturing device 408 through said transparent window 316 of said modular incubator chamber 300, once said modular incubator chamber 300 is being docked in said docking port 402.

In an embodiment of the modular incubator system according to the second aspect of the present invention and in respect of one or more of said one or more modular incubator chambers 300, said transparent window 316 of said modular incubator chamber 300 is arranged at a bottom part 357 of said housing 302.

In this embodiment the image capturing device 408 will accordingly be arranged in the docking port 402 at a lower portion focusing in an upward direction.

In an embodiment of the modular incubator system according to the second aspect of the present invention and in respect of one or more of said one or more modular incubator chambers 300, said transparent window 316 of said housing 302 of said modular incubator chamber is having an elongate shape, such as an elongate and linear extension extending in a direction Y transversal to said longitudinal direction X of said housing of said modular incubation chamber 300.

Hereby the image capturing device may capture images of a plurality of viable biological materials being accommodated in the same culture dish and arranged in a line having a direction Y which is transversal to said longitudinal direction X of said housing of said modular incubation chamber 300.

In an embodiment of the modular incubator system according to the second aspect of the present invention and in respect of one or more specific docking ports 402 of said docking station 400, said specific docking port comprises its own dedicated image capturing device 408 which is configured to only capture images relating to a modular incubator chamber 300 which is being docked in said specific docking port 402.

In an embodiment of the modular incubator system according to the second aspect of the present invention and in respect of a number N of adjacently arranged docking ports 402 of said docking station 400, said adjacently arranged docking ports share a common image capturing device 408 in the sense that one and only one image capturing device is responsible for capturing images relating to a modular incubator chamber 300 which is being docked in one of said N adjacently arranged docking ports 402, wherein said docking station comprises a displacement device 482, such as an electrically driven and remotely controlled displacement device 482 for enabling displacement of said common image capturing device 408 in relation to said N adjacently arranged docking ports 402 of said docking station 400.

Hereby one image capturing device is responsible for the capturing of images of biological materials being accommodated in different modular incubator chambers which are being docked in different docking port 402 of the docking station 400.

In an embodiment said number N is being an integer selected in the ranges of 2-25 or more, such as 4-22, for example 6-20, such as 8-18, such as 10-16 or 12-14.

Independently, one or more image capturing devices 408, preferably all image capturing devices 408 of the docking station 400 may comprise or be coupled to a displacement device 482, such as an electrically driven and remotely controlled displacement device 482 for enabling displacement of said common image capturing device 408 in a direction transversal to the longitudinal direction X of a modular incubator chamber 300 being docked in a docking port 402 with the view to enable such capturing device 408 to focus on more than one culture well in a culture dish 310 being accommodated in the interior of the modular incubator chamber 300, wherein such culture wells are arranged in such direction transversal to the longitudinal direction X.

In an embodiment of the modular incubator system according to the second aspect of the present invention and in respect of one or more of said one or more modular incubator chambers 300 and in respect of one or more of said one or more docking ports 402 of said docking station 400, said modular incubator chamber 300 is being configured to be docked in said docking port 402 with its first end 340 facing said docking port 402.

In an embodiment of the modular incubator system according to the second aspect of the present invention and in respect of one or more of said one or more modular incubator chambers 300, said modular incubator chamber 300, in the interior 306 thereof, comprises a light source 372 for directing light to the area of the culture dish support 308 of said modular incubator chamber 300, thereby enabling illumination of a viable biological material in a situation of capturing images of said viable biological material.

In an embodiment said light source 372 is being attached to said lid 304 of the housing 302 of said modular incubator chamber 300, at an inner side thereof.

Hereby light can easily be directed to a viable biological material which is arranged at a lower part of the interior 306 of the modular incubator chamber 300.

In an embodiment said light source 372 is being selected from the group of one or more LEDs, one or more laser diodes, one or more incandescent light bulbs.

In an embodiment of the modular incubator system according to the second aspect of the present invention and in respect of one or more of said one or more modular incubator chambers 300, said culture dish support 308 is defining a planar support surface for supporting said culture dish 310.

In an embodiment of the modular incubator system according to the second aspect of the present invention and in respect of one or more of said one or more modular incubator chambers 300, said housing 302 of said modular incubator chamber 300, such as at an outer portion thereof, is being provided with electric connectors 322 for providing electric power and/or electric signals to said modular incubator chamber; and wherein in respect of one or more docking ports 402 of said docking station 400, said docking port is being provided with electric connectors 410.

Hereby conveying of electric power or electric signals between said docking port 402 and said modular incubator chamber 300 is enabled.

In an embodiment of the modular incubator system according to the second aspect of the present invention and in respect of one or more of said one or more modular incubator chambers 300, said lid 304 is being a hinged lid which is being connected to said housing of said modular incubator chamber via a hinge.

In an embodiment of the modular incubator system according to the second aspect of the present invention and in respect of one or more of said one or more modular incubator chambers 300, said housing 302 of said modular incubator chamber 300 comprises a display 324 which is being configured to display information relating to an operational status of the incubation taking place in said modular incubator chamber.

In an embodiment of the modular incubator system according to the second aspect of the present invention the docking station 400 comprises said docking ports 402 in an arrangement of one or more shelves of adjacently positioned docking ports 402, wherein in case said docking station comprises two or more shelves, said shelves are being arranged above each other.

In an embodiment of the modular incubator system according to the second aspect of the present invention and in respect of one or more of said one or more modular incubator chambers 300, said modular incubator chamber comprises an incubation chamber engagement means 326 and wherein in respect of one or more docking ports 402 of said docking station 400, said docking port comprises a docking port engagement means 414, wherein said incubation chamber engagement means 326 is being configured to enter into engagement with said docking port engagement means 414.

Hereby, easy and proper positioning and optionally also fixing said modular incubator chamber 300 in said docking port 402, as well as detaching said modular incubator chamber 300 from said docking port 402 of said docking station 400 is provided.

In an embodiment of the modular incubator system according to the second aspect of the present invention the modular incubator system 500 comprises an image processing unit 660 for image processing of images captured by said image capturing device(s) 408, wherein said modular incubator system 400 furthermore comprises a data storage 658 for storing images captured by said image capturing units 408 and/or for storing images processed by said image processing unit.

An image processing unit is beneficial for manipulating the images captured, such as for adjusting contrast, for filtering and for generating time-lapse series of images.

In an embodiment of the modular incubator system according to the second aspect of the present invention one or more of said image capturing devices 408 of said docking ports 402 of said docking station is/are being coupled to said image processing unit 660.

In an embodiment of the modular incubator system according to the second aspect of the present invention and in respect of one or more of said modular incubator chambers 300 said valve(s) 2,4 is being arranged with its front end 10,20 pointing outward; and wherein in respect of one or more of said docking ports 402 said valve(s) 4,2 is being arranged with its front end 20,10 pointing outward.

In an embodiment of the modular incubator system according to the second aspect of the present invention and in respect of one or more of said one or more modular incubator chambers 300, a first valve 2 of said valve system 100 is being arranged in said chamber inlet opening for gas 312 and in said chamber outlet opening for gas 314; and wherein in respect of one or more of said one or more docking station 402 of said docking station 400, a second valve 4 of said valve system 100 is being arranged in said docking port outlet opening for gas 404 and in said docking port inlet opening for gas 406;
or
wherein in respect of one or more of said one or more modular incubator chambers 300, a second valve 4 of said valve system 100 is being arranged in said chamber inlet opening for gas 312 and in said chamber outlet opening for gas 314; and wherein in respect of one or more of said one or more docking station 402 of said docking station 400, a first valve 2 of said valve system 100 is being arranged in said docking port outlet opening for gas 404 and in said docking port inlet opening for gas 406.

Accordingly, two valves 2 of the valve system 100 may be arranged in the modular incubator chamber 300 and two valves 4 of the valve system 100 may be arranged in one or more of the docking ports 402 of the docking station 400 of the modular incubator system 500, or alternatively, two valves 4 of the valve system 100 may be arranged in the modular incubator chamber 300 and two valves 2 of the valve system 100 may be arranged in one or more of the docking ports 402 of the docking station 400 of the modular incubator system 500.

In an embodiment of the modular incubator system according to the second aspect of the present invention, the image capturing device 408 comprises microscopic optics so as to enable capturing of microscope images.

Hereby magnified images may be captured which improves study of the morphological nature of the biological materials being incubated.

In an embodiment of the modular incubator system according to the second aspect of the present invention and in respect of one or more of said modular incubator chambers 300, said modular incubator chamber comprises in its interior 306 an electric heating element 318 for heating the interior of said modular incubator chamber, and wherein said modular incubator chamber comprises a power source 320 for providing power to said heating element 318, wherein said electric heating element 318 is being electrically connected to said power source 320.

In an embodiment said power source 320 is being an electric power source, such as a battery, for example a rechargeable battery.

In an embodiment said heating element 318 is being thermally connected to a heat distribution element for distributing heat dissipated in said heating element; wherein said heat distribution element is being arranged, at least partly, in the interior 306 of said modular incubator chamber 300.

In an embodiment said chamber comprises a thermostat 374 and an electric thermostatic circuit 376, wherein said electric heating element 318, said power source 320 and said thermostat 374 are being electrically connected in said electric thermostatic circuit 376 so as to enable thermostatic control of the temperature inside said modular incubator chamber 300.

The above embodiments provide for upholding a desirable and predetermined and optionally also optimum temperature in the interior 306 of the modular incubator chamber 300 in a situation where the modular incubator chamber is removed from its associated docking port 402 with the view to perform visual inspection and manual replenishing, removal, or exchange of growth medium to the biological materials being incubated.

In an embodiment of the modular incubator system according to the second aspect of the present invention, the number of modular incubator chambers 300 of said modular incubator system 500 is selected from the ranges 1-100, such as 2-95, for example 5-90, e.g. 10-85, such as 15-80, for example 20-75, e.g. 25-70, 30-65, such as 35-60, e.g. 40-55 or 45-50.

In an embodiment of the modular incubator system according to the second aspect of the present invention, the number of docking ports 402 in said docking station 400 of said modular incubator system 500 is selected from the ranges 1-100, such as 2-95, for example 5-90, e.g. 10-85, such as 15-80, for example 20-75, e.g. 25-70, 30-65, such as 35-60, e.g. 40-55 or 45-50.

In an embodiment of the modular incubator system 500 according to the second aspect of the present invention and in respect of one or more of said docking ports 402 of said docking station 400 of said modular incubator system 500, preferably in respect of all said docking ports 402, said docking port outlet opening for gas 404 comprises a flow restrictor for restricting the magnitude of flow of gas flowing into said docking port 402.

In one embodiment the flow restrictor may comprises a tube through which the gas is conveyed to said docking port 402, wherein said tube optionally is having a cross-sectional area selected from the ranges of 0.2-8 mm², such as 0.5-7 mm², for example 1-6 mm², such as 2-5 mm² or 3-4 mm²; and/or the length of said tube is optionally selected from the ranges of 5-30 mm, such as 8-25 mm, for example 10-22 mm, e.g. 15-20 mm.

Such a flow restrictor aids in balancing the flow of gas through the docking ports 402 comprising a modular incubator chamber 300 with the capacity of the gas supply system 200 and thereby also aids in making the flow of gas through the different docking ports 402 equal to each other.

In an embodiment of the modular incubator system according to the second aspect of the present invention the docking station 400 comprises a gas distribution system 204 for supplying gas to and from one or more of said one or more docking ports 402, wherein said gas distribution system 204 comprises a main gas supply line 210 and a main gas return line 212, wherein in respect of one or more of said docking ports 402, said docking port inlet opening for gas 404 is being fluidly connected to said main gas supply line 210, and said docking port outlet opening for gas 406 is being fluidly connected to said main gas return line 212.

In one embodiment said gas distribution system 204 comprises a number of manifold pairs 214, wherein each manifold pair comprises an inlet manifold 216 and an outlet manifold 218, wherein said inlet manifold 216 is being fluidly connected to said main gas supply line 210 and wherein said outlet manifold 218 is being fluidly connected to said main gas return line 212; wherein each manifold pair 214 is connected to one or more docking ports 402 of said docking station 400 in such a way that in respect of a specific manifold pair 214, and in respect of said one or more docking ports 402 being connected thereto, said docking port outlet opening for gas 404 of said docking port 402 is being fluidly connected to said inlet manifold 216, and said docking port inlet opening for gas 406 of said docking port 402 is being fluidly connected to said outlet manifold 218.

In one embodiment said docking station 400 comprises a gas supply system 200, wherein said gas supply system 200 comprises a gas source 202 and said gas distribution system 204, wherein said gas source comprises a supply gas outlet 206 and a return gas inlet 208, wherein said supply gas outlet 206 of said gas source 202 is being fluidly connected to said main gas supply line 210 of said gas distribution system 204, and wherein said return gas inlet 208 of said gas source 202 is being fluidly connected to said main gas return line 212 of said gas distribution system 204.

In these embodiments comprising a gas distribution system 204 it is possible to supply gas from a gas source 202 to the docking ports 402 via the main gas supply line 210 and to return gas from the docking ports to the gas source 202 via the main gas return line 212.

In an embodiment of the modular incubator system according to the second aspect of the present invention, the gas source 202 of said gas supply system 200 comprises a gas mixing box 242 comprising said supply gas outlet 206 and said return gas inlet 208 of said gas source, wherein said main gas supply line 210 of said gas distribution system 204 is being fluidly connected to said supply gas outlet 206, and wherein said main gas return line 212 of said gas distribution system 204 is being fluidly connected to said return gas inlet 208 of said gas source 202, thereby forming a flow loop 244 comprising said gas distribution system 204 and said gas mixing box 242; wherein said flow loop comprises a pump 246.

Hereby circulating gas in said loop, and also through the gas distribution system of the docking station is possible.

The purpose of the gas source is to provide and deliver a desired gas composition to the gas distribution system 204, including the various docking ports 402 of the docking station 400.

In one embodiment of this embodiment the pump 246 is being arranged downstream in relation to said main gas return line 212.

In one embodiment the flow loop 244 comprises a pump oscillation damper 247, wherein said pump oscillation damper optionally is being arranged immediately downstream in relation to said pump 246.

The pump oscillation damper will equalize small and rapid pressure variations caused by each pump stroke of the pump.

In an embodiment of the modular incubator system according to the second aspect of the present invention, the flow loop 244 comprises a pressure sensor, such as a differential pressure sensor 248 for sensing the pressure of gas supplied to said main gas supply line 210 of said gas distribution system 204, wherein said pressure senor 248 optionally is being arranged immediately upstream in relation to said main gas supply line 210 of said gas distribution system 204.

The pressure sensor 248 allows for regulating the pump 246 on order to maintain a desired pressure in the flow loop 244.

In one embodiment the pressure sensor 249 is being a differential pressure sensor, sensing a pressure relative to the pressure of the return gas inlet 208.

In one embodiment the flow loop 244 comprises a release valve 249 for enabling pressure relief in said flow loop, wherein said release valve optionally is being arranged immediately downstream in relation to said main gas return line 212 of said gas distribution system 402.

The pressure release valve 249 enables improved control of the pressure in the flow loop 344.

In an embodiment of the modular incubator system according to the second aspect of the present invention, the gas mixing box 242 comprises an inlet for N₂ gas 250; and an inlet for CO₂ gas 251, wherein said inlet for N₂ gas 250 is fluidly connected to an N₂ valve 252 for regulating the inflow of N₂, and an N₂ mass flow sensor 253 arranged downstream of said N₂ valve 252 for sensing the amount of N₂ flowing into said gas mixing box 242; and wherein said inlet for CO₂ gas 251 is fluidly connected to a CO₂ valve 254 for regulating the inflow of CO₂, and an CO₂ mass flow sensor 255 arranged downstream of said CO₂ valve 254 for sensing the amount of CO₂ flowing into said gas mixing box 242.

Herby it is possible to control the inlet of N₂ gas and the inlet of CO₂ gas into the gas mixing box 242 with the view to obtain a desired, predetermined and optimum gas composition in the gas mixing box 242.

In an embodiment of the modular incubator system according to the second aspect of the present invention, the flow loop 244 comprises a mass flow sensor 256 arranged at an upstream position in relation to said gas mixing box 242 for sensing the amount of return gas entering said gas mixing box.

Information relating to the amount of return gas entering said gas mixing box is used for determining the total amount of N₂ gas and CO₂ gas which needs to be introduced into the gas mixing box 242.

In an embodiment of the modular incubator system according to the second aspect of the present invention, the gas source 202 comprises an O₂ sensor 258 for sensing the concentration of O₂ exiting said gas distribution system 204; and wherein said gas source 202 comprises a CO₂ sensor 260 for sensing the concentration of CO₂ exiting said gas distribution system 204, wherein said O₂ sensor and/or said CO₂ sensor optionally is/are being arranged downstream in relation to said pump 246.

Information relating to the concentration of O₂ and the concentration of CO₂ exiting said gas distribution system 204 is used for determining the specific amount of N₂ gas and the specific amount of CO₂ gas which needs to be introduced into the gas mixing box 242.

In an embodiment of the modular incubator system according to the second aspect of the present invention, the gas source 202 comprises a temperature sensor 262 for sensing the temperature of gas circulating in said flow loop 244, wherein said temperature sensor optionally is being arranged downstream in relation to said pump 246, preferably at a position corresponding to the position of said O₂ sensor 258.

In an embodiment of the modular incubator system according to the second aspect of the present invention, the gas source 202 comprises a pressure sensor 264 for sensing the absolute pressure in said flow loop 244 wherein said pressure sensor optionally is being arranged downstream in relation to said pump 246, preferably at a position corresponding to the position of said CO₂ sensor 260.

The temperature sensor 262 and pressure sensor 264 are useful for performing compensation of the readings of the O₂ sensor 258 due to temperature sensitivity thereof and the readings of the CO₂ sensor 260 due to sensitivity thereof towards pressure.

In an embodiment of the modular incubator system according to the second aspect of the present invention, the flow loop 244 comprises a UV sanitizer 266 for sanitizing gas flowing in said flow loop 244 via electromagnetic radiation in the UV range, wherein said UV sanitizer optionally being arranged immediately downstream in relation to said main gas return line 212.

In an embodiment of the modular incubator system according to the second aspect of the present invention, the gas source 202 comprises one or more filters 268, such as HEPA and/or VOCs filters, wherein such a filter is being arranged immediately upstream in relation to said main gas supply line 210, and/or wherein such a filter is being arranged immediately upstream in relation to the inlet for N₂ gas 250 into said gas mixing box 242; and/or wherein such a filter is being arranged immediately upstream in relation to the inlet for CO₂ gas 251 into said gas mixing box 242.

In an embodiment of the modular incubator system according to the second aspect of the present invention, the gas source 202 comprises a gas mixing control system 270, wherein said gas mixing control system is electrically connected to one or more of the following sensors for receiving sensing signals therefrom: said N₂ mass flow sensor 253 for sensing the amount of N₂ flowing into said gas mixing box; said CO₂ mass flow sensor 255 for sensing the amount of CO₂ flowing into said gas mixing box; said mass flow sensor 256 for sensing the amount of return gas entering said gas mixing box; said O₂ sensor 258 for sensing the concentration of O₂ exiting said main gas return line 212 of said gas distribution system 204; said CO₂ sensor 260 for sensing the concentration of CO₂ exiting said main gas return line 212 of said gas distribution system 204; said temperature sensor 262 for sensing the temperature circulating in said flow loop 244; said pressure sensor 264 for sensing an absolute pressure in said flow loop 244, said pressure sensor 248 for sensing the pressure of gas supplied to said gas main gas supply line 210 of said distribution system 204.

This embodiment enables gaining information of various parameters which are to be used in providing a feed back when controlling the operation of the gas source 202.

In an embodiment of the modular incubator system according to the second aspect of the present invention, the gas mixing control system 270 is electrically connected to one or more of the following elements for control thereof: said N₂ valve 252 for regulating the inflow of N₂ into said gas mixing box 242; said CO₂ valve 254 for regulating the inflow of CO₂ to said gas mixing box 242; said pump 246 for circulating gas in said flow loop 244; said release valve 249.

This embodiment enables providing a feed back when controlling the operation of the gas source 202.

In one embodiment the gas mixing control system 270 is being configured to receive input from said pressure sensor 248 and on the basis thereof control said pump 246, optionally also to activate said release valve 249 in order to maintain a desired and predetermined pressure of gas supplied to said main gas supply line 210 of said gas distribution system 204.

Hereby the pressure in the flow loop 244 can be controlled.

In an embodiment of the modular incubator system according to the second aspect of the present invention, the gas mixing control system 270 is being configured to receive input from said mass flow sensor 256, and on the basis on said input to determine the total amount of CO₂ gas and N₂ gas needed to be supplied via said inlet for CO₂ gas 251 and via said inlet for N₂ gas 250 according to desired and predetermined criteria.

In an embodiment of the modular incubator system according to the second aspect of the present invention, the gas mixing control system 270 is being configured to receive input from said CO₂ sensor 260 and said O₂ sensor 258, and on the basis of the CO₂ concentration sensed, is configured to control said CO₂ valve 254, by transmitting a control signal thereto, and thereby regulating the inflow of CO₂ gas in order to reach a desired and predetermined CO₂ concentration, and wherein subsequently, said gas mixing control system 270 on the basis of the O₂ concentration sensed, is configured to control said N₂ valve 252, by transmitting a control signal thereto, and thereby regulating the inflow of N₂ gas in order to reach a desired and predetermined O₂ concentration.

In an embodiment of the modular incubator system according to the second aspect of the present invention, the gas mixing control system 270 is configured to use the input from said temperature sensor 262 for compensating the temperature sensitivity of said O₂ sensor 258. In an embodiment of the modular incubator system according to the second aspect of the present invention, the gas mixing control system 270 is configured to use the input from said pressure sensor 264 for compensating the pressure sensitivity of said CO₂ sensor 260.

In an embodiment of the modular incubator system according to the second aspect of the present invention, the gas mixing control system 270 is being configured to maintain a pressure of gas supplied to said main gas supply line 210 of said gas distribution system 204, relative to the ambient atmospheric pressure, of 3-20 mbar, such as 5-18 mbar, such as 10-15 mbar above that ambient atmospheric pressure.

In an embodiment of the modular incubator system according to the second aspect of the present invention, the gas mixing control system 270 is being configured to maintain a CO₂ concentration of gas entering said main gas supply line 210 of said gas distribution system 204 in the range of 5-10%, such as 6-9% or 7-8%; and/or an O₂ concentration of gas entering said main gas supply line 210 of said gas distribution system 204 in the range of 5-10%, such as 6-9% or 7-8%.

In an embodiment of the modular incubator system according to the second aspect of the present invention said modular incubator system comprises a control unit 650 for controlling the operation thereof.

In an embodiment of the modular incubator system according to the second aspect of the present invention said control unit 650 is being coupled to an input device 652, such as an alphanumerical input device for allowing a user to provide settings input relating to a desired operational protocol of said modular incubator system.

In an embodiment of the modular incubator system according to the second aspect of the present invention said control unit 650 is being coupled to a display unit 654 for displaying, to a user, information relating to settings and/or operational status of said modular incubator system 500.

In an embodiment of the modular incubator system according to the second aspect of the present invention and in respect of one or more docking ports 402 of said docking station 400, and or in respect of a modular incubator chamber 300 being docked therein, said control unit 650 is being configured for independently controlling one or more of the following: the setting of said thermostat 374 of a modular incubator chamber 300 being docked therein, switching on and off an active light source 372 of a modular incubator chamber 300 being docked therein and/or regulating the intensity of light emitting from that active light source 372, said gas mixing control system 270; said image capturing unit 408 and/or said associated displacement device 482 of one or more of said docking ports 402 of the docking station 400 of the modular incubator system 500; said image processing unit 660.

Hereby the operation of the modular docking system 500 can easily be controlled centrally.

In an embodiment of the modular incubator system according to the second aspect of the present invention said control unit 650 is being coupled to a data processing unit 656 and optionally also to a data storage 658 for aiding in handling information during controlling of said modular incubator system.

In an embodiment of the modular incubator system according to the second aspect of the present invention said control unit 650 is being configured for conducting automatic operation of said modular incubator system 500 by independently controlling of one or more of the following: the setting of said thermostat 374 of a modular incubator chamber 300 being docked in a docking port 402, switching on and off an active light source 372 of a modular incubator chamber 300 being docked in a docking port 402 and/or regulating the intensity of light emitting from that active light source 372 of a modular incubator chamber being docked in a docking port 402, said gas mixing control system 270; said image capturing unit 408 and/or said associated displacement device 482 of one or more of said docking ports 402 of the docking station 400 of the modular incubator system 500, said gas mixing control system 270 of said docking station 400 according to predefined control instructions provided thereto, said image processing unit 660.

In an embodiment of the modular incubator system according to the second aspect of the present invention said control unit 650 is being configured for generating time lapse capturing of images by said image capturing device(s) 408.

### The Third Aspect of the Present Invention

In a third aspect the present invention relates to a modular incubator chamber 300, wherein said modular incubator chamber 300 comprises a housing 302 having a first end 340 and a second end 342, thereby defining a longitudinal direction X between said first end and said second end;
wherein said housing comprises a lid 304, wherein said lid is being configured to be able to shift between an open configuration allowing access to the interior 306 of said modular incubator chamber and a closed configuration, sealing off access to the interior of said modular incubator chamber;
wherein said modular incubator chamber 300, at said interior 306 thereof, comprises a culture dish support 308 for positioning a culture dish 310 with the view to accommodate one or more biological materials M within the housing 302 of said modular incubator chamber 300;
wherein said housing of said modular incubator chamber 300 comprises a chamber inlet opening for gas 312, wherein said chamber inlet opening for gas 312 is being in fluid connection with the interior 306 of said modular incubator chamber; wherein one valve 2,4 of the valve system 100 according to the first aspect according to the present invention is being arranged in said chamber inlet opening for gas 312;
wherein said housing 302 of said modular incubator chamber furthermore comprises a chamber outlet opening for gas 314, wherein said chamber outlet opening for gas 314 is being in fluid connection with the interior 306 of said modular incubator chamber; wherein one valve 2,4 of the valve system 100 according to the first aspect according to the present invention is being arranged in said chamber outlet opening for gas 314.

In an embodiment of the modular incubator chamber according to the third aspect of the present invention said modular incubator chamber 300 is comprising features as defined in respect of the modular incubator chamber 300 of the modular incubator system 500 according to according to the first aspect according to the present invention.

### The Fourth Aspect of the Present Invention

In a fourth aspect the present invention relates to a docking station 400, wherein said docking station comprises one or more docking ports 402 for receiving a modular incubator chamber 300;
wherein in respect of one or more docking ports 402 of said docking station 400, said docking port 402 comprises a docking port outlet opening for gas 404; thereby enabling transfer of gas from said docking port 402 of said docking station 400 to an interior 302 of said modular incubator chamber 300 via said docking port outlet opening for gas 404; wherein one valve 4,2 of the valve system 100 according to the first aspect according to the present invention is being arranged in said docking port outlet opening for gas 404; and
wherein said docking port 402 furthermore comprises a docking port inlet opening for gas 406, thereby enabling transfer of gas from the interior 306 of a modular incubator chamber 300 to said docking port 402 of said docking station 400; wherein one valve 4,2 of the valve system 100 according to the first aspect according to the present invention is being arranged in said docking port inlet opening for gas 406.

In an embodiment of the docking station according to the fourth aspect of the present invention said docking station is comprising features as defined in respect of the docking station of the modular incubator system 500 according to the first aspect according to the present invention.

### The Fifth Aspect of the Present Invention

In a fifth aspect the present invention provides a use of a valve system 100 according to the first aspect according to the present invention in a modular incubator system 500.

### The Sixth Aspect of the Present Invention

In a sixth aspect the present invention provides a use of a modular incubator system 500 according to the second aspect of the present invention, for incubating a viable biological material.

In an embodiment said biological material is being an oocyte or an embryo, such as a human oocyte or a human embryo.

### The Seventh Aspect of the Present Invention

In a seventh aspect the present invention provides a use of a modular incubator chamber 300 according to the third aspect of the present invention, for incubating a viable biological material.

In an embodiment said biological material is being an oocyte or an embryo, such as a human oocyte or a human embryo.

### The Eighth Aspect of the Present Invention

In an eighth aspect the present invention provides a use of a docking station 400 according to the fourth aspect of the present invention, for incubating a viable biological material.

In an embodiment said biological material is being an oocyte or an embryo, such as a human oocyte or a human embryo.

### The Ninth Aspect of the Present Invention

In a ninth aspect the present invention method related to a method of incubating a viable biological material, wherein said method comprises:
i) providing a modular incubator system 500 according to the first aspect of the present invention;
ii) providing a viable biological material;
iii) arranging said viable biological material in a culture dish 310 and subsequently arranging said culture dish in the interior 306 of a modular incubator chamber 300 of said modular incubator system 500;
iv) docking said modular incubator chamber 300 in a docking port 402 of said docking station 400 of said modular incubator system 500;
v) allowing said viable biological material to be incubated in said modular incubator chamber 300;
vi) supplying gas into and out of the interior 306 of said chamber via said valve system 100 of said modular incubator system 500.

In an embodiment the method further comprising the step of:
viii) removing said incubator chamber 300 from said docking port 402 of said docking station 400, when desired, in order to manually inspect the viable biological material, and optionally also to remove, add or exchange growth medium/media in said culture dish 310.

It is noted that in the amended claims relating to the third aspect of the present invention, viz. the modular incubator chamber, reference is made to that features of this modular incubator chamber may be as defined in respect of the claims relating to the second aspect of the present invention, viz, the modular incubator system.

This shall be construed to mean that embodiment of the modular incubator chamber per se may be as defined in the claims relating to embodiments of the modular incubator system.

This shall also be construed to mean that to the extent that an interrelationship between the modular incubator chamber and the docking station or a docking port thereof is defined in such embodiments relating to the modular incubator system, the corresponding embodiment of the modular incubator chamber, claimed by reference to the modular incubator system, shall be considered suitable to enter into such interrelationship.

Likewise, it is noted that in the amended claims relating to the fourth aspect of the present invention, viz, the docking station, reference is made to that features of this docking station may be as defined in respect of the claims relating to the second aspect of the present invention, viz, the modular incubator system.

This shall be construed to mean that embodiment of the docking station per se may be as defined in the claims relating to embodiments of the modular incubator system.

This shall also be construed to mean that to the extent that an interrelationship between the modular incubator chamber and the docking station or a docking port thereof is defined in such embodiments relating to the modular incubator system, the corresponding embodiment of the docking station, claimed by reference to the modular incubator system, shall be considered suitable to enter into such interrelationship.

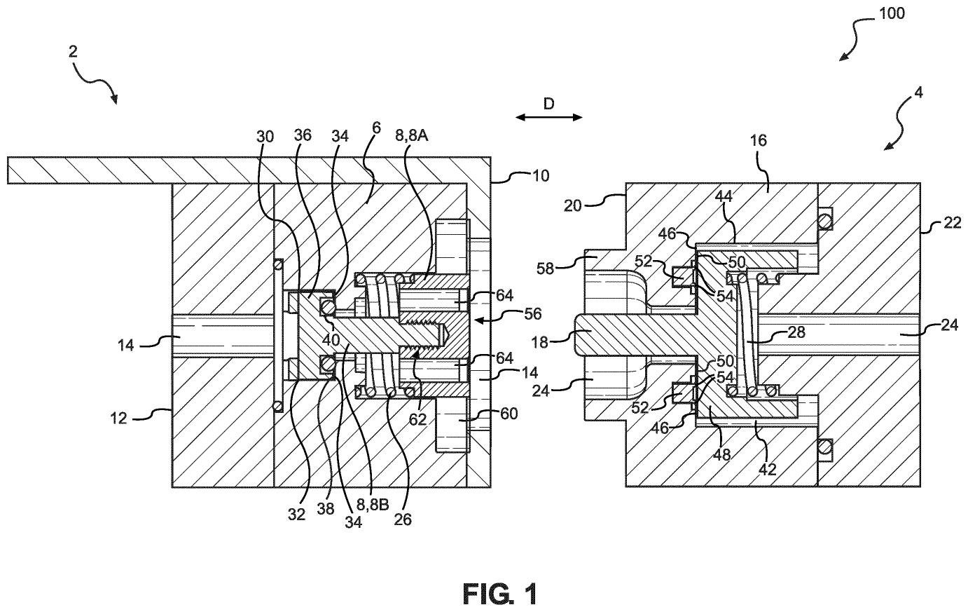

Referring now to the figures for better illustrating the present invention FIG. 1 is a cut-through cross-sectional view of the valve system of the first aspect of the invention showing the two valves being separated from each other.

FIG. 1 illustrates the valve system 100 comprising a first valve 2 and a second valve 4.

The first valve 2 comprises a first valve body 6 and a first valve element 8. The first valve body 6 comprises a front end 10 and a rear end 12 and the first valve body 6 comprises a first throughgoing channel 14 extending between the front end 10 and the rear end 12 of the first valve body 6.

The first valve element 8 is being arranged in the first throughgoing channel 14 of the first valve body 6 in such a way that said first valve element 8 is being displaceable in the displacement direction D between a first extreme position and a second extreme position in said first throughgoing channel 14.

In the first extreme position, the first valve element 8 is being displaced in a direction towards the front end 10 (i.e. to the right in FIG. 1) of said first valve body 6, and in the second extreme position, the first valve element 8 is being displaced in a direction towards the rear end 12 of said first valve body 6 (i.e. to the left in FIG. 1).

In valve 2 of FIG. 1, the valve element 8 of the first valve 2 is in its first extreme position.

The dimensions and geometries of the first valve body 6 and the first valve element 8 are adapted to each other in such a way that, once being positioned in the first extreme position, the first valve element 8 will block passage through said first throughgoing channel 14 between said front end 10 and said rear end 12 thereof, and in such a way, that once being displaced in a direction towards the second extreme position, the first valve element 8 will provide passage through the first throughgoing channel 14 between said front end 10 and said rear end 12 thereof.

It is seen that the first valve 2 comprises a first spring 26. The first spring is adapted to interact with the first valve element 8, relative to the first valve body 6, in such a way the first spring 26 will displace the first valve element 8, when not otherwise acted upon, towards the first extreme position thereof (to the right), thereby closing the first valve 2. Accordingly, when not engaged from any external force, the first valve 2 will be closed.

It is seen in FIG. 1 that once being in said first extreme position, a first contact surface 38 of a widened portion 36 of the first valve element 8 will be in close contact with a first inclined surface portion 34 of a first wall segment 32 of a widened portion 30 of the first through-going channel 14. Hereby passage through the first throughgoing channel 14 between the front end 10 and the rear end 12 thereof is blocked.

Moreover, as explained below, once being displaced towards the second extreme position, the first contact surface 38 of the widened portion 36 of the first valve element 8 of the first valve element 8 will have lost contact with the first inclined surface portion 34 of the first wall segment 32 of a widened portion 30 of the first through-going channel 14. Hereby passage through the first throughgoing channel 14 between the front end 10 and the rear end 12 thereof is enabled.

The first inclined surface portion 34 of the first wall segment 32 of a widened portion 30 of the first through-going channel 14 is being inclined relative to the direction D of displacement of the first valve element 8 in the first through-going channel 14.

A first valve gasket 40 which is in the form of an O-ring provides for a tight seal between the valve element 8 and the first inclined surface portion 34 of the first wall segment 32 of the first valve body of the first valve 2.

The second valve 4 of the valve system 100 comprises a second valve body 16 and a second valve element 18. The second valve body 16 comprising a front end 20 and a rear end 22 and the second valve body 16 comprises a second throughgoing channel 24 extending between that front end 20 and that rear end 22 of said second valve body 16.

The second valve element 18 is being arranged in the second throughgoing channel 24 of the second valve body 16 in such a way that the second valve element 18 is being displaceable in a displacement direction D between a first extreme position and a second extreme position in said second throughgoing channel 24.

In the first extreme position, the second valve element 18 is being displaced in a direction towards the front end 20 (i.e. to the left in FIG. 1) of the second valve body, and in said second extreme position, the second valve element 18 is being displaced in a direction towards the rear end 22 of the second valve body 16 (i.e. to the right in FIG. 1).

The dimensions and geometries of the second valve body 16 and the second valve element 18 are adapted to each other in such a way that, once being positioned in the first extreme position, the second valve element 18 will block passage through the second throughgoing channel 24 between the front end 20 and said rear end 22 thereof, and in such a way, that once being displaced in a direction towards said second extreme position, the second valve element 18 will provide passage through said second throughgoing channel 24 between said front end 20 and said rear end 22 thereof.

In valve 4 of FIG. 1, the valve element 18 of the second valve 4 is in its first extreme position. It is seen that the second valve 4 comprises a second spring 28. The second spring is adapted to interact with the second valve element 18, relative to the second valve body 16, in such a way the second spring 28 will displace the first valve element 18, when not otherwise acted upon, towards the first extreme position thereof (to the left), thereby closing the second valve 4. Accordingly, when not engaged from any external force, the second valve 4 will be closed.

FIG. 1 shows that once being in the first extreme position, a second contact surface 50 of a widened portion 48 of the second valve element 18 will be in close contact with a second inclined surface portion 46 of a wall segment 44 of a widened portion 42 of the second through-going channel 24. Hereby passage through the second throughgoing channel 24 between the front end 20 and the rear end 22 thereof is blocked.

Moreover, as explained below, once being displaced towards the second extreme position, the second contact surface 50 of the widened portion 48 of the second valve element 18 will have lost contact with the second inclined surface portion 46 of the wall segment 44 of the widened portion 42 of the second through-going channel 24. Hereby, passage through said second throughgoing channel 24 between the front end 20 and the rear end 22 thereof is enabled.

The second inclined surface portion 46 of the wall segment 44 of a widened portion 42 of the second through-going channel 24 is being inclined relative to the direction D of displacement of said second valve element 24.

Second valve gaskets 52 provide for a tight seal between the valve element 18 and the second inclined surface portion 46 of the wall segment 44 of the through-going channel 24 of the second valve body 16 of the second valve 4.

The second valve gaskets 52 comprise lip portions 54 in the form of tapered lip portions, pointing towards the second contact surface 50 of the widened portion 48 of the second valve element 18.

The lip portions 52 provides better sealing between the valve element 18 and the second inclined surface portion 46 of the wall segment 44 of the second throughgoing channel 24.

The dimensions and geometries of the first valve element 8 and the second valve element 18 are being adapted to each other in such a way that upon bringing the first valve 2 into contact with the second valve 4, by making their respective front ends 10,20 approach each other, the second valve element 18 of the second valve 4 is configured to displace the first valve element 8 of the first valve 2 towards the second extreme position thereof, thereby opening the first valve 2, and further, the first valve element 8 of the first valve 2 is configured to displace the second valve element 18 of the second valve 4 towards the second extreme position thereof, thereby opening the second valve 4.

This is further explained below.

FIG. 1 also illustrates that the valve body 6 of the first valve 2, at the front end 10 thereof, comprises a depression 56, and that the valve body 16 of the second valve 4, at the front end 20 thereof, comprises a hollow protrusion 58 surrounding at least part of the second valve element 18 of the second valve 4.

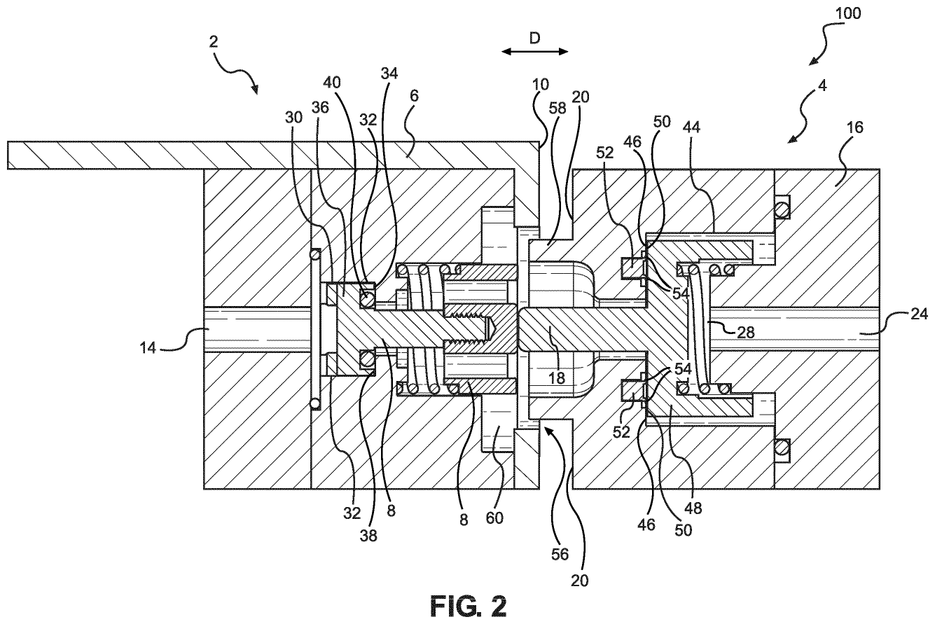

The dimensions and the geometry of the depression 56 and the protrusion 58 are adapted to each other in such a way that the protrusion 58 of the second valve body 16 will fit into the depression 56 of the first valve body 6. This is illustrated in FIG. 2.

FIG. 1 also shows that the valve body 6 at the front end 10 thereof and at the end of the depression 46, comprises an end gasket 60 which surrounds at least part of the first valve element 8 and the first through-going channel 14 of the first valve 2, thereby allowing the protrusion 58 of the second valve body 16 of the second valve 4 to abut the end gasket 60, when the protrusion 58 of the second valve body 16 of the second valve 4 is being inserted into the depression 56 of the first valve body 6 of the first valve 2. Hereby leaking of gas can be avoided or at least considerably reduced.

Finally, FIG. 1 shows that the first valve element 8 comprises a first part 8a and a second part 8b, wherein the first part 8a of the first valve element 8 is being arranged proximate to the front end 10 of the first valve body 6, and wherein the second part 8b of the first valve element 8 is being arranged distal to the front end 10 of said the valve body 6.

The first part 8a and the second part 8b of the first valve element 8 are being connected to each other via a threaded tap/threaded hole arrangement 62.

Hereby adjustment of the total length of the first valve element 8, in a direction parallel to the direction of displacement D of said first valve element 8 is possible. This feature allows for adjusting the extent to which the first valve element 8 extends at the front end 10 of the body 6 of the first valve 2.

The first valve element 8, at the end proximate to the front end 10 of the first valve body 6, comprises through-going holes 64 for allowing conveying of gas through the holes into the first throughgoing channel 15 of said first valve body 6.

FIG. 2 is a cut-through cross-sectional view of the valve system of FIG. 1 in which the two valves are shown in a situation of just touching each other.

FIG. 2 shows a situation where the first valve 2 and the second valve 4 have approached each other by bringing their respective front ends 10,20 together in such a way that the first valve element 8 of the first valve 2 just touches the second valve element 18 of the second valve 4.

Hereby, as seen, the hollow protrusion 58 of the second valve body 16 has been partly inserted into the depression 56 of the first valve body 6 of the first valve. In FIG. 2 the two valves 2 and 4 are still in a closed configuration.

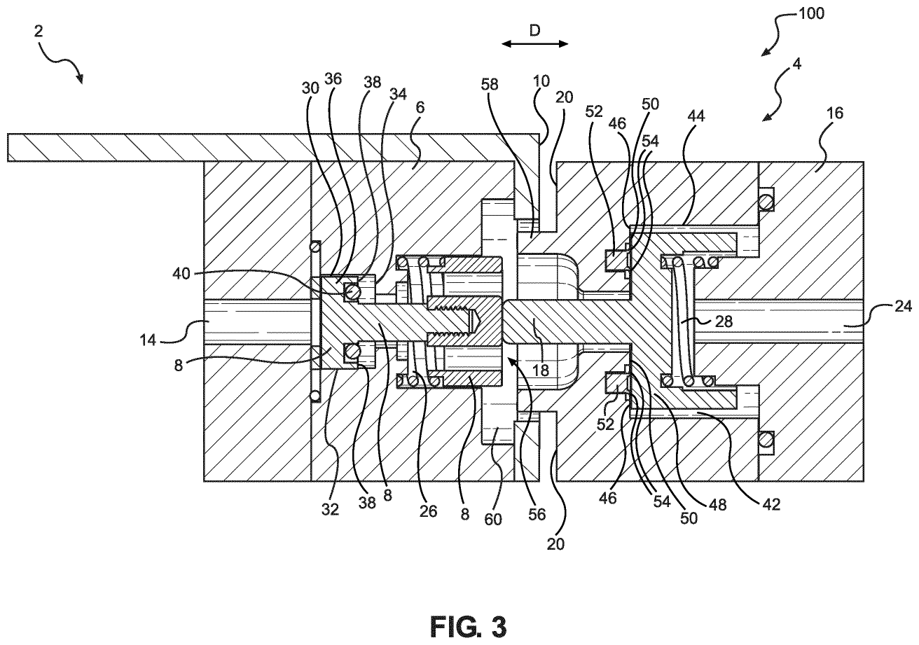

FIG. 3 is a cut-through cross-sectional view of the valve system of FIG. 1 in which the two valves are shown in a situation so close that they start to open.

In FIG. 3 the first valve body 6 of the first valve 2 and the second valve body 16 of the second valve 4 have further approached so that the hollow protrusion 48 of the second valve body 16 now touches the end gasket 60.

This extension of insertion of the hollow protrusion 58 has made the second valve element 18 of the second valve 4 displace the first valve element 8 of the first valve 2 towards the second extreme position (i.e. to the left in FIG. 3).

Thereby, first contact surface 38 of the widened portion 36 of the first valve element 8 has lost contact with the first inclined surface portion 34 of the wall first segment 32 of the widened portion 30 of the first throughgoing channel 14. This in turn allows passage of gas through the first throughgoing channel 14 in the first valve body 6 between the front end 10 and the rear end 12 thereof.

As the spring constant of the second spring 28 is larger than the spring constant of the first spring 26, the second valve element 18 of the second valve 4 has not yet been displaced.

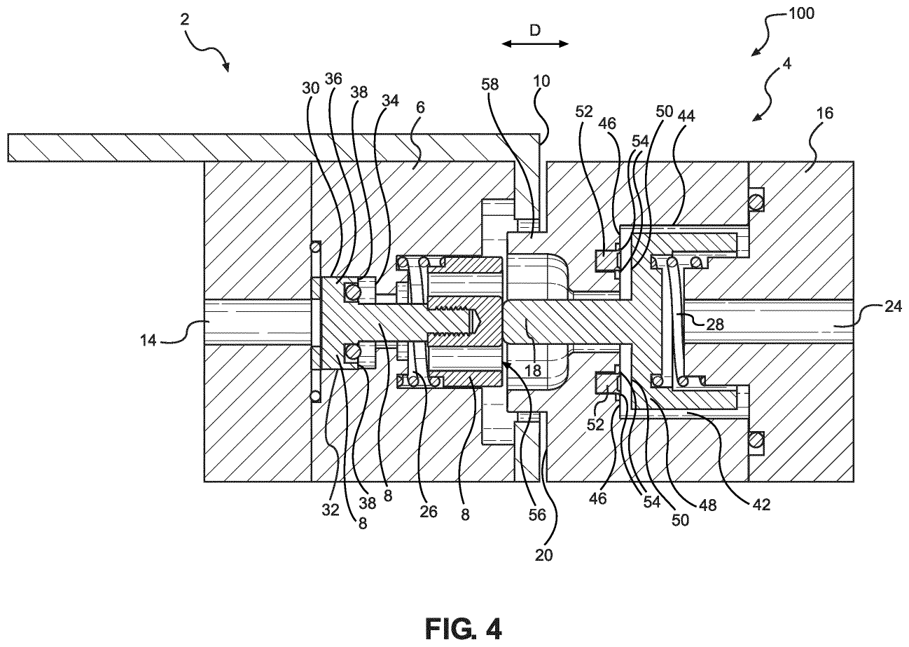

FIG. 4 is a cut-through cross-sectional view of the valve system of FIG. 1 in which the two valves have been fully engaged so that they are both open.

FIG. 4 shows that the first valve body 6 of the first valve 2 and the second valve body 16 of the second valve 4 have fully engaged so that the hollow protrusion 58 of the second valve body 16 now is being pressed into the end gasket 60.

Hereby the first valve element 8 of the first valve 2 has displaced the second valve element 18 of the second valve 4 towards the second extreme position thereof (i.e. to the right in FIG. 4).

Also, the second contact surface 50 of the widened portion 48 of the second valve element 18 has lost contact with the second inclined surface portion 46 of the second wall segment 44 of the widened portion 42 of the second throughgoing channel 24. Hereby passage of gas through the second throughgoing channel 24 in the second valve body 16 between the front end 20 and the rear end 22 is enabled.

Accordingly, in FIG. 4 both the first valve 2 and the second valve 4 are open which means that once in the configuration illustrated in FIG. 4, the valve system 100 will be able to convey gas through the respective throughgoing channel 14,24 of the two valves bodies 6,16 from a rear end 22 of the second valve body 16 to the rear end 12 of the first valve body 6 or in the opposite direction.

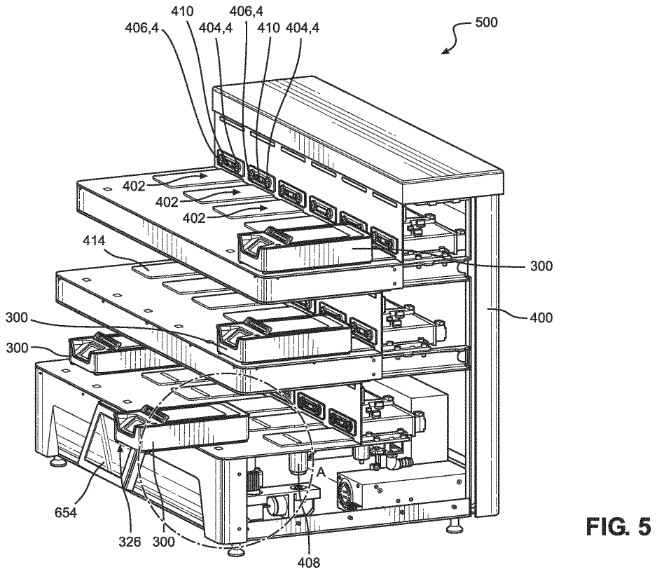

The valve system 100 illustrated in FIGS. 1-4 is for use in a modular incubator system. This is further illustrated in FIG. 5.

FIG. 5 is a perspective view illustrating a modular incubator system according to the second aspect of the present invention.

The modular incubator system 500 in FIG. 5 comprises a plurality of modular incubator chambers 300 in combination with a docking station 400.

The docking station 400 shown in FIG. 5 comprises three shelves each comprising six docking ports 402. Each docking port comprises second engagement means 414 for engaging with corresponding first engagement means 326 of the modular incubator chamber 300 to be docked in the docking port 402.

FIG. 5 shows that each docking port 402 comprises a docking port outlet opening for gas 404 and a docking port inlet opening for gas 406. The openings 404 and 406 each comprises a valve 4 of the valve system 100 according to the first aspect of the present invention.

Hereby is achieved that gas can be supplied from the docking port 402 to a modular incubator chamber 300 being docked into the docking port, and also that gas can be returned from the modular incubator chamber into the docking port.

Also seen in FIG. 5 is that the docking ports 402 comprises an electric connector 410 for supplying electric power from the docking port to a modular incubator chamber 300 being docked into that docking port. Alternatively or additionally, the electric connector 410 may convey electric signals between the docking port 402 and the modular incubator chamber 300.

FIG. 5 also illustrates that below the shelf comprising the docking ports an image capturing device 408 is arranged in such a way that this image capturing device is configured for capturing images of a biological material being accommodated in a culture dish 310 which is resting on the culture dish support 308 in the interior 306 of the modular incubator chamber 300 being docked above the image capturing unit 408.

Hereby, morphological changes of a viable biological material can be monitored while incubating that biological material in a modular incubator chamber 300 which is being docked in a docking port 402 in a docking station 400 of the modular incubator system 500 and while a desired gaseous atmosphere is being maintained in the interior 306 of the modular incubator chamber.

The image capturing device comprises microscope optics for capturing close-up images.

The image capturing device(s) 408 may be configured for automatically capturing of images of the biological material being incubated in a modular incubator chamber 300

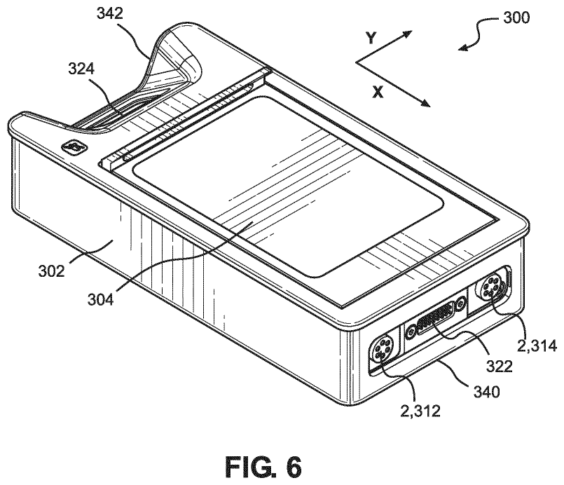

FIG. 6 is a perspective view showing a modular incubator chamber of the docking system of the second aspect of the present invention. The modular incubator chamber is also subject of the third aspect of the present invention.

FIG. 6 shows the modular incubator chamber 300 for incubating a viable biological material. The modular incubator chamber comprises a housing 302 and the housing comprises a lid 304 which is being configured to be able to shift between an open configuration allowing access to the interior 306 of the modular incubator chamber and a closed configuration, sealing off access to the interior 306 of said modular incubator chamber.

The housing 302 of the modular incubator chamber comprises a chamber inlet opening for gas 312 which is in fluid connection with the interior 306 of the modular incubator chamber, thereby allowing supplying gas into said chamber via said chamber inlet opening for gas 312.

The chamber inlet opening for gas comprises a first valve 2, which comprises the features as disclosed above in respect of the valve system 100 of the first aspect of the invention.

The housing 302 of said modular incubator chamber 300 furthermore comprises a chamber outlet opening for gas 314, which is being in fluid connection with the interior of the modular incubator chamber, thereby allowing conveying gas out of the chamber 300 via the chamber outlet opening for gas 314. The chamber outlet opening for gas comprises a first valve 2 which comprises the features as disclosed above in respect of the valve system 100 of the first aspect of the invention.

By providing the housing 302 of the modular incubator chamber 300 with a chamber inlet opening for gas 312 and an associated first valve 2 and by providing the modular incubator chamber 300 with a chamber outlet opening for gas 314 and an associated first valve 2 it is possible to convey a gas having a suitable and desired gas composition into the interior of the modular incubator chamber 300 from a docking port 402 of a docking station 400, as will be further explained below, and further it is possible to make the gas in the interior of the modular incubator chamber exit the interior of the chamber through the chamber outlet opening for gas 314 and its associated first valve 2 and return to the docking station 402.

Hereby a constant supply of gas having an optimum chemical composition can be delivered to the interior 306 of the chamber 300. This will ensure optimum incubation conditions in terms of gaseous composition of the environment in the interior 306 of the chamber 300 when incubating a biological material.

Moreover, with the incubator system according to the second aspect of the present invention it is possible to conduct a relatively large number of parallelly conducted incubations under similar conditions in the individual modular incubator chambers, while altering only one parameter from one modular incubator chamber to another. The difference in development of the viable biological material being incubated in the various modular incubator chambers can then be assigned to the one incubation parameter that is altered from one modular chamber to the other.

This allows for determining optimum incubation conditions of an embryo or an oocyte being incubated.

FIG. 6 also shows that the housing 302 of the modular incubator chamber 300 comprises a display 324 which is being configured to display information relating to details of the incubation taking place in said modular incubator chamber and that the housing 302 at a first end 340 thereof, is being provided with electric connectors 322 for providing electric power to the modular incubator chamber or for conveying electric signals between the modular incubator chamber 300 and the docking port 402 of the docking station 400.

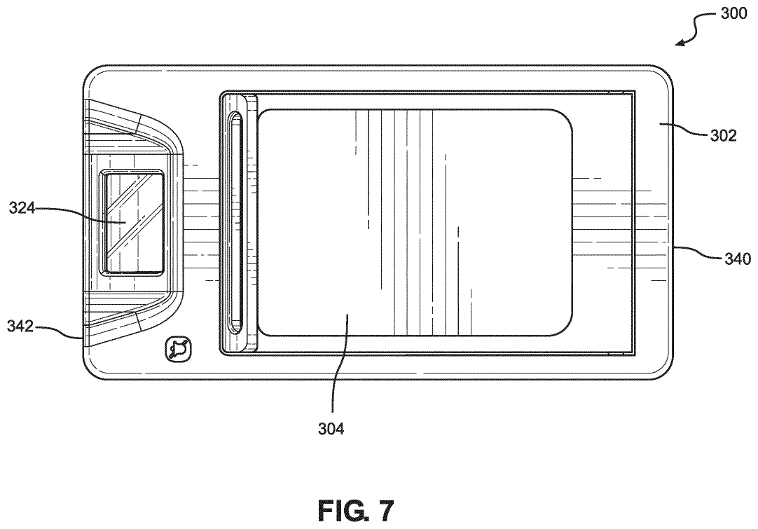

FIG. 7 is a plan top view of the modular incubator chamber 300 illustrated in FIG. 5.

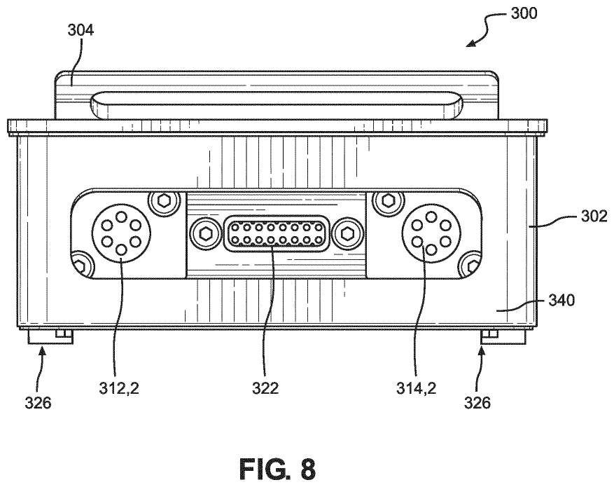

FIG. 8 is a plan rear view of the modular incubator chamber 300 illustrated in FIGS. 5 and 6 as seen from its first end.

FIG. 8 shows that the modular incubator chamber 300 comprises a chamber engagement means 326. These first engagement means 326 are configured to enter into engagement with a docking port engagement means 414 in a docking port 402 of a docking station 400.

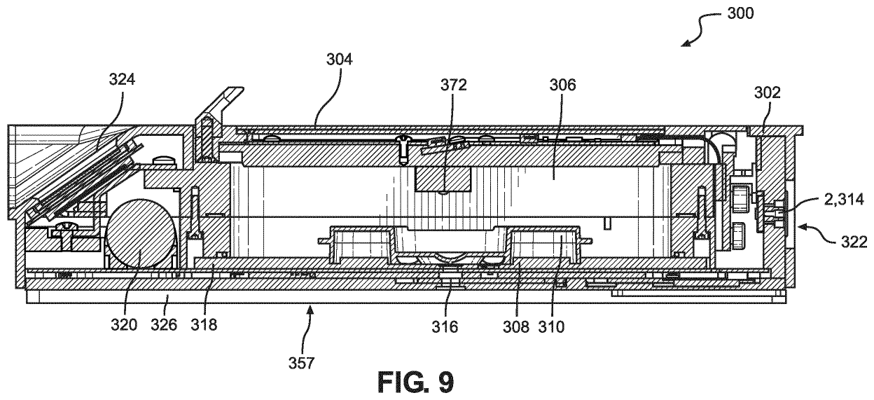

FIG. 9 is a cross-sectional view of the modular incubator chamber 300 illustrated in FIGS. 6, 7 and 8.

FIG. 9 shows that the housing 302 of the modular incubator chamber 300 comprises a transparent window 316 for allowing capturing of images of a biological material being accommodated in the interior thereof, trough said transparent window. As seen the window is arranged at the bottom 357 of the housing 302 of the modular incubator chamber 300.

The modular incubator chamber 300 also comprises, in its interior 306, an electric heating element 318 for heating the interior of said modular incubator chamber. The modular incubator chamber also comprises a power source 320 in the form of a rechargeable battery for providing power to said heating element 318 which is being electrically connected to the power source 320. A light source 372 is attached to an inner side of the lid 304 of the modular incubator chamber 300.

As seen in FIG. 9 the interior 306 of the modular incubator chamber 300 comprises a culture dish support 308 for positioning a culture dish 310. Hereby, one or more biological materials can be accommodated and incubated within the housing 302 of the modular incubator chamber 300.

Also seen in FIG. 9 is the chamber engagement means 326 which is being adapted to engage with docking port engagement means 414 of the docking port 402 into which the modular incubator chamber 300 is to be docked.

When such a proper positioning of the of the modular incubator chamber 300 in the docking port 402 has been attained via the chamber engagement means 326 of the chamber 300 and via the docking port engagement means 414 of the docking port 402, the relative position of the two electric connectors 410 and 322 of the docking port and the modular incubator chamber, respectively, and the two inlet openings 312 and 406 with their respective valves 2,4 and the two outlet openings 314,404 with their respective valves 2,4 will match pairwise so as to allow electric connection between the connectors 410 and 322. Likewise, the gas openings 312, 404 and, 314,406 will match pairwise so that passage of gas from the docking port outlet opening for gas 404 into the interior 306 of the modular incubator chamber 300 via the modular incubator chamber inlet opening for gas 312 and the valves 2,4 of the valve system 100 is enabled, and so that passage of gas from the interior 306 of the modular incubator chamber 300 is possible via the modular incubator chamber outlet opening for gas 314 and into the docking port inlet opening for gas 406 and via the valves 2,4 of the valve system 100 is enabled.

Accordingly, the modular docking system 500 of the present invention allows for continuously providing gas into the interior 306 of the modular incubator chamber from a gas source 412.

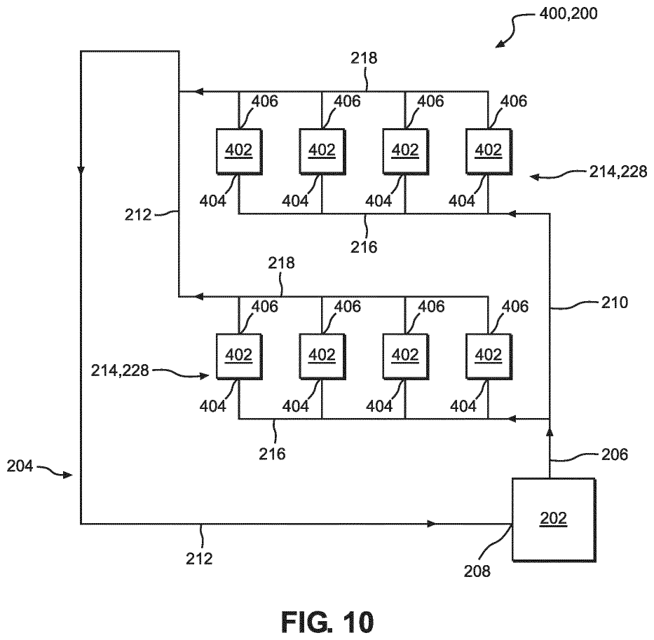

This is further illustrated in FIG. 10.

FIG. 10 is a diagram illustrating the concept of the gas supply system which in incorporated in the docking station of the modular incubator system of the present invention.

FIG. 10 shows a gas supply system 200 to be used with a docking station 400 of the modular incubator system 500 according to the present invention. The gas supply system 200 comprises a gas source 202 and a gas distribution system 204.

The gas distribution system 204 comprises a plurality of docking ports 402 each having a docking port outlet opening for gas 404 and a docking port inlet opening for gas 406.

In respect of all the docking ports, the docking port outlet openings for gas 404 are in fluid connection with an inlet manifold 216 and the docking port inlet openings for gas 406 are in fluid connection with an outlet manifold 218.

A main gas supply line 210 supplies gas from a supply gas outlet 206 of the gas source 202 to the inlet manifolds 216, and a main gas return line 212 returns gas from the outlet manifolds 218 to a return gas inlet 208 of the gas source 202.

Thereby gas can be circulated from the gas source 202 via the gas distribution system 204 to the docking ports 402 and back to the gas source 202.

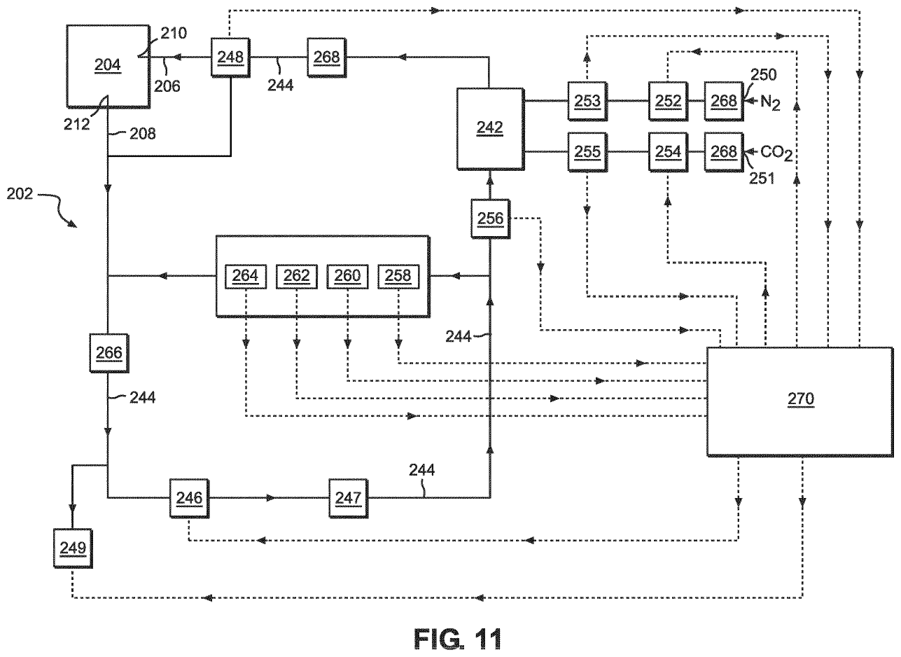

In order to secure a desired and predetermined and optimum gas composition of the gas supplied to the docking stations, the gas source is provided with specific features as disclosed with reference to FIG. 11.

FIG. 11 is a diagram illustrating one embodiment of a design of a gas supply system comprising a gas source to be used with the docking station of the present invention.

In FIG. 11 solid lines represent flow lines for gas, whereas dashed lines represent signal lines for conveying electric signals or electric power.

FIG. 11 shows the gas distribution system 204 comprising its main gas supply line 210 and its main gas return line 212 (illustrated with the rectangle in upper left corner).

The main gas supply line 210 and the main gas return line 212 of the gas distribution system 204 is fluidly connected to a gas source 202 as described below.

The gas source 202 of said gas supply system 200 comprises a gas mixing box 242 connected to the supply gas outlet 206 and the return gas inlet 208 of the gas source.

The main gas supply line 210 of the gas distribution system 204 is being fluidly connected to the supply gas outlet 206, and the main gas return line 212 of the gas distribution system 204 is being fluidly connected to the return gas inlet 208 of the gas source 202.

Hereby a flow loop 244 comprising the gas distribution system 204 and the gas mixing box 242 is formed. The flow loop 244 comprises a pump 246 for circulating gas in that loop.

It is seen that the pump 246 is being arranged downstream in relation to the main gas return line 212. Also seen in FIG. 11 is that the flow loop 244 comprises a pump oscillation damper 247, which is being arranged immediately downstream in relation to the pump 246.

Moreover, the flow loop 244 comprises a pressure sensor in the form of a differential pressure sensor 248 for sensing the pressure of gas supplied to the main gas supply line 210, relative to the pressure in the return gas inlet line 208 of the gas distribution system 204. The pressure senor 248 is being arranged immediately upstream in relation to the main gas supply line 210 of the gas distribution system 204.

The flow loop 244 further comprises a release valve 249 for enabling pressure relief in the flow loop. The release valve is arranged immediately downstream in relation to the main gas return line 212 of the gas distribution system 402.

Also seen in FIG. 11 is that the gas mixing box 242 comprises an inlet for N₂ gas 250; and an inlet for CO₂ gas 251.

The inlet for N₂ gas 250 is fluidly connected to an N₂ valve 252 for regulating the inflow of N₂, and an N₂ mass flow sensor 253 arranged downstream of the N₂ valve 252 for sensing the amount of N₂ flowing into said gas mixing box 242.

The inlet for CO₂ gas 251 is fluidly connected to a CO₂ valve 254 for regulating the inflow of CO₂, and an CO₂ mass flow sensor 255 arranged downstream of the CO₂ valve 254 for sensing the amount of CO₂ flowing into the gas mixing box 242.

The flow loop 244 also comprises a mass flow sensor 256 arranged at an upstream position in relation to the gas mixing box 242 for sensing the amount of return gas entering the gas mixing box.

It is seen that the gas source 202 comprises an O₂ sensor 258 for sensing the concentration of O₂ exiting the gas distribution system 204; and that the gas source 202 comprises a CO₂ sensor 260 for sensing the concentration of CO₂ exiting the gas distribution system 204.

The O₂ sensor and the CO₂ sensor is arranged downstream in relation to the pump 246.

A temperature sensor 262 for sensing the temperature of gas circulating in said flow loop 244 is included in the gas source 202. The temperature sensor is arranged downstream in relation to the pump 246 at a position corresponding to the position of the O₂ sensor 258.

A pressure sensor 264 for sensing the absolute pressure in the flow loop 244 is included in the gas source 202. This pressure sensor is arranged downstream in relation to the pump 246, at a position corresponding to the position of the CO₂ sensor 260.

Also seen in FIG. 11 is that the flow loop 244 comprises a UV sanitizer 266 for sanitizing gas flowing in the flow loop 244 via electromagnetic radiation in the UV range. The UV sanitizer is arranged immediately downstream in relation to the main gas return line 212.

It is seen in FIG. 11 that the gas source 202 comprises filters 268 in the form of HEPA/VOCs filters. One such a filter is arranged immediately upstream in relation to the main gas supply line 210. Another such a filter is arranged immediately upstream in relation to the inlet for N₂ gas 250 into the gas mixing box 242; and a third such a filter is being arranged immediately upstream in relation to the inlet for CO₂ gas 251 into the gas mixing box 242.

Finally, it is seen in FIG. 11 that the gas source 202 comprises a gas mixing control system 270.

It is seen that the gas mixing control system 270 is electrically connected to one or more of the following sensors for receiving sensing signals therefrom: the N₂ mass flow sensor 253 for sensing the amount of N₂ flowing into the gas mixing box; the CO₂ mass flow sensor 255 for sensing the amount of CO₂ flowing into the gas mixing box; the mass flow sensor 256 for sensing the amount of return gas entering the gas mixing box; the O₂ sensor 258 for sensing the concentration of O₂ exiting the main gas return line 212 of the gas distribution system 204; the CO₂ sensor 260 for sensing the concentration of CO₂ exiting the main gas return line 212 of the gas distribution system 204; the temperature sensor 262 for sensing the temperature circulating in the flow loop 244; the pressure sensor 264 for sensing an absolute pressure in the flow loop 244, the pressure sensor 248 for sensing the pressure of gas supplied to the gas main gas supply line 210 of the distribution system 204.

Also seen in FIG. 11 is that the gas mixing control system 270 is electrically connected to one or more of the following elements for control thereof: the N₂ valve 252 for regulating the inflow of N₂ into the gas mixing box 242; the CO₂ valve 254 for regulating the inflow of CO₂ to the gas mixing box 242; the pump 246 for circulating gas in the flow loop 244; the release valve 249.

The control of the gas source by the gas mixing control system 270 is performed in accordance with two control regimes. The first control regime relates to controlling the pressure of gas exiting the supply gas outlet 206, and the second control regime relates to controlling the concentration of CO₂ and O₂ of gas exiting the supply gas outlet 206. The two control regimes are conducted concurrently. This is further explained below.

The gas mixing control system 270 is being configured to receive input from the pressure sensor 248 and on the basis thereof control the pump 246 and optionally also activate the release valve 249 in order to maintain a desired and predetermined pressure of gas supplied to the main gas supply line 210 of the gas distribution system 204.

The gas mixing control system 270 is further configured to receive input from the mass flow sensor 256, and on the basis on this input to determine the total amount of CO₂ gas and N₂ gas needed to be supplied via the inlet for CO₂ gas 251 and via the inlet for N₂ gas 250 according to desired and predetermined criteria.

Based on the information relating to the total amount of CO₂ gas and N₂ gas needed to be supplied, as described above, the gas mixing control system 270 will be able to determine the mutual proportion of the CO₂ gas and N₂ gas to be supplied to the gas mixing box 242. This is performed by receiving input from the CO₂ sensor 260 and the O₂ sensor 258.

On the basis of the CO₂ concentration sensed, the gas mixing control system 270 will control the CO₂ valve 254, by transmitting a control signal thereto, and thereby regulate the inflow of CO₂ gas in order to reach a desired and predetermined CO₂ concentration.

Subsequently, the gas mixing control system 270 will on the basis of the O₂ concentration sensed, control the N₂ valve 252, by transmitting a control signal thereto, and thereby regulate the inflow of N₂ gas in order to reach a desired and predetermined O₂ concentration.

By using the gas source as disclosed above, a constant circulation of gas will be supplied to one or more modular incubator chamber 300 being docked in a respective docking port 402 of the docking station 400. By constantly regulating the inflow of CO₂ gas and N₂ gas based on sensed concentration of CO₂ and O₂ in the return gas from the gas distribution system 204, an optimum and predetermined gas composition can be maintained.

Due the design of the gas distribution system 204, an equal magnitude of flow through each modular incubator chamber 300 can be upheld, thereby minimizing variations of the composition of gas in the modular incubator chambers 300 from one chamber to another even though a modular incubator chamber 300 has been removed from its docking port 402.

It should be noted that when reference is made to an upstream position relative to another position, that upstream position is construed to mean a position still within the gas source 202 and preferably not so much upstream that it passes the gas mixing box 242 or the gas distribution system 204.

Likewise, when reference is made to a downstream position relative to another position, that downstream position is construed to mean a position still within the gas source 202 and preferably not so much downstream that it passes the gas mixing box 242 or the gas distribution system 204.

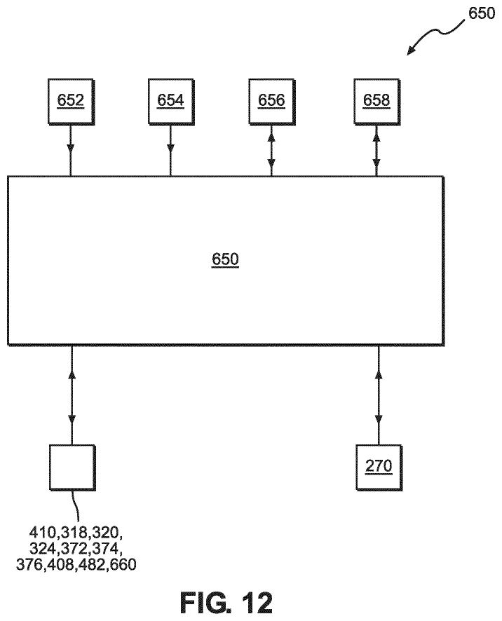

FIG. 12 is a diagram illustrating the working mode of the controlling of the modular incubator system according to the invention.

FIG. 12 shows the control unit 650 for controlling the operation of the modular incubator system 500. The control unit is coupled to an input device 652 in the form of an alphanumerical input device for allowing a user to provide settings input relating to a desired operational protocol of said modular incubator system.

A display unit 654 for displaying, to a user, information relating to settings and/or operational status of one or more of the modular incubator chambers 300 which via a docking port 402 is coupled to the control unit 650.

It is seen that the control unit 650 is coupled to a electric connectors 410 of the docking ports 402 of the docking station 400. Thereby electrical power and electric signals can be provided to one or more modular incubator chambers 300 which is/are being docked into a docking port 402 of the docking station 400 of the modular incubator system 500 via the associated connector 322 of the modular incubator chamber 300.

By being connected to the docking ports 402 of the docking station 400 it will be thereby possible, when one or more modular incubator chambers 300 is/are docked into a docking port 402 of the docking station 400, and by using the control unit 650, to independently control one or more of the following: the setting of said thermostat 374 of a modular incubator chamber 300 being docked therein, switching on and off an active light source 372 of a modular incubator chamber 300 being docked therein and/or regulating the intensity of light emitting from that active light source 372, the gas mixing control system 270; the image capturing unit 408 and/or the associated displacement device 482 of one or more of the docking ports 402 of the docking station 400 of the modular incubator system 500; and also the image processing unit 660.

The control unit 650 may comprises a CPU or other data processor 656 for processing the information involved in controlling the operation of the modular incubator system 500, e.g. by involving a computer program for handing the information involved in the controlling of the operation and the control unit 650 may also comprise a data storage 658.

Thereby automatic operation of the modular incubator system 500 may be performed in the sense that control unit 650 may inter alia independently control the temperature, the gas composition, the switching on and off of the light source 372, and the image capturing unit 408 of one or more of the modular incubator chambers.

Accordingly, with the modular incubator system 500 of the present invention, viable biological materials can be incubated in one or more modular incubators 300 which are being docked in docking ports 402 of the docking station 400, and at the same time visual monitoring of the biological material can be conducted via the image capturing device 408.

Moreover, at the same time a desired gas composition can be maintained in the interior 306 of each modular incubator chamber 300. As the modular incubator chambers comprises valves 2,4 in the respective inlet opening for gas 312 and outlet opening for gas 314, the gas atmosphere will be maintained and not disturbed by the outside atmosphere (relative to the interior 306 of the modular incubator chamber 300), even when the modular incubator chamber is removed from its respective docking port 402 of the docking station 400 of the modular incubator system 500. Upon such removal of the modular incubator chamber 300 from a docking port 402 of the docking station 400, the power source 320 and the electric heating element 318 will enable upholding the temperature in the interior 306 of the modular incubator chamber 300.

The present invention thereby allows for incubation of a biological material in the modular incubator chamber 300 and at the same time allows for visual monitoring of the morphological development of the biological material, while minimizing the detrimental effects involved in deviating from an optimized and desired gaseous atmosphere in the interior of the modular incubator chamber.

It should be noted that in respect of a number N of adjacently arranged docking ports 402 of the docking station 400, these adjacently arranged docking ports 402 may share a common image capturing device 408 in the sense that one and only one image capturing device is responsible for capturing images relating to a modular incubator chamber 300 which is being docked in one of these N adjacently arranged docking ports 402.

In such a situation, a displacement device 482 in the form of an electrically motorized suspension of the image capturing device 408 is being configured to be displaced, upon receiving a signal thereto, along a displacement track extending below these N number of adjacent docking ports 402 for enabling displacement of that common image capturing device 408 in relation to the N adjacently arranged docking ports 402 of the docking station 400.

Thereby the common image capturing device 408 will be able to capture images of a biological material being accommodated in the interior 306 of a modular incubator chamber 300 being docked in any of said N docking ports 402 of the docking station 400.

Although the above embodiments have been disclosed in a way where the first valve 2 of the valve system 100 is arranged in the modular incubator chamber 300 and in such a way that the second valve 4 of the valve system 100 is arranged in the docking port 402, the opposite arrangement may also be possible. Both in respect of valves 2,4 letting gas into the chamber 300 and in respect of valves 2,4 letting gas out the chamber, or both.

It should be understood that all features and achievements discussed above and in the appended claims and clauses in relation to one aspect of the present invention and embodiments thereof apply equally well to the other aspects of the present invention and embodiments thereof.

The present invention may be defined according to the following clauses:

Clause 1. A valve system (100) for a modular incubator system (500), wherein said valve system comprises a first valve (2) and a second valve (4);
wherein said first valve (2) comprises:
   a first valve body (6); and
   a first valve element (8);
wherein said first valve body (6) comprising a front end (10) and a rear end (12);
wherein said first valve body (6) comprises a first throughgoing channel (14) extending between said front end (10) and said rear end (12) of said first valve body (6);
wherein said first valve element (8) is being arranged in said first throughgoing channel (14) of said first valve body (6) in such a way that said first valve element (8) is being displaceable in a displacement direction D between a first extreme position and a second extreme position in said first throughgoing channel (14), wherein in said first extreme position, said first valve element (8) is being displaced in a direction towards the front end (10) of said first valve body (6), and wherein in said second extreme position, said first valve element (8) is being displaced in a direction towards the rear end (12) of said first valve body (6);
wherein the dimensions and geometries of said first valve body (6) and said first valve element (8) are adapted to each other in such a way that, once being positioned in said first extreme position, said first valve element (8) will block passage through said first throughgoing channel (14) between said front end (10) and said rear end (12) thereof, and in such a way, that once being displaced in a direction towards said second extreme position, said first valve element (8) will provide passage through said first throughgoing channel (14) between said front end (10) and said rear end (12) thereof;
wherein said second valve (4) comprises:
   a second valve body (16); and
   a second valve element (18);
wherein said second valve body (16) comprising a front end (20) and a rear end (22);
wherein said second valve body (16) comprises a second throughgoing channel (24) extending between said front end (20) and said rear end (22) of said second valve body (16);
wherein said second valve element (18) is being arranged in said second throughgoing channel (24) of said second valve body (16) in such a way that said second valve element (18) is being displaceable in a displacement direction D between a first extreme position and a second extreme position in said second throughgoing channel (24), wherein in said first extreme position, said second valve element (18) is being displaced in a direction towards the front end (20) of said second valve body (16), and wherein in said second extreme position, said second valve element (18) is being displaced in a direction towards the rear end (22) of said second valve body (16);
wherein the dimensions and geometries of said second valve body (16) and said second valve element (18) are adapted to each other in such a way that, once being positioned in said first extreme position, said second valve element (18) will block passage through said second throughgoing channel (24) between said front end (20) and said rear end (22) thereof, and in such a way, that once being displaced in a direction towards said second extreme position, said second valve element (18) will provide passage through said second throughgoing channel (24) between said front end (20) and said rear end (22) thereof.

Clause 2. A valve system (100) according to clause 1, wherein the dimensions and geometries of said first valve element (8) and said second valve element (18) are being adapted to each other in such a way that upon bringing said first valve (2) into contact with said second valve (4), by making their respective front ends (10,20) approach each other, said second valve element (18) of said second valve (4) is configured to displace said first valve element (8) of said first valve (2) towards the second extreme position thereof, thereby opening said first valve (2), and further, said first valve element (8) of said first valve (2) is configured to displace said second valve element (18) of said second valve (4) towards the second extreme position thereof, thereby opening said second valve (4).

Clause 3. A valve system (100) according to clause 1 or 2, wherein said first valve (2) comprises a first spring (26), wherein said first spring is adapted to interact with said first valve element (8), relative to said first valve body (6), in such a way that said first spring (26) will displace said first valve element (8), when not otherwise acted upon, towards said first extreme position thereof, thereby closing said first valve (2);
and/or wherein
said second valve (4) comprises a second spring (28), wherein said second spring is adapted to interact with said second valve element (18), relative to said second valve body (16), in such a way that said second spring (28) will displace said second valve element (18), when not otherwise acted upon, towards said first extreme position thereof, thereby closing said second valve (4).

Clause 4. A valve system (100) according to clause 3, wherein said first valve (2) comprises said first spring (26) having first spring constant and wherein said second valve (4) comprises said second spring (28) having a second spring constant, wherein said first spring constant is equal to said second spring constant, thereby making said first valve (2) and said second valve (4) open approximately simultaneous upon being brought into contact with each other, or wherein said first spring constant is smaller than said second spring constant, thereby making said first valve (2) open before said second valve (4), upon being brought into contact with each other; or wherein said first spring constant is larger than said second spring constant, thereby making said second valve (4) open before said first valve (2), upon being brought into contact with each other.

Clause 5. A valve system (100) according to any of the clauses 1-4, wherein said first throughgoing channel (14) of said first valve 2 comprises a widened portion (30) having a first wall segment (32) defining a first inclined surface portion (34) which is being inclined relative to the direction of displacement D of said first valve element (8), and wherein said first valve element (8) comprises a widened portion (36) having a first contact surface (38), wherein said widened portion (36) of said first valve element (8) is being accommodated in said widened portion (30) of said first through-going channel (14) in such a way that when said first valve element (8) is being in its first extreme position, said first contact surface (38) of said first valve element (8) is being in contact with said first inclined surface portion (34) of said first throughgoing channel (14), thereby rendering said first valve (2) closed by blocking passage through said first through-going channel (14); and in such a way that when said first valve element (8) is being in its second extreme position, said first contact surface (38) of said first valve element (8) is being separated from said first inclined surface portion (34) of said first throughgoing channel (14), thereby rendering said first valve (2) open by providing passage through said first through-going channel (14).

Clause 6. A valve system (100) according to clause 5, wherein said first contact surface (38) of said first valve element (8) is being inclined relative to the direction of displacement D of said first valve element (8).

Clause 7. A valve system 100 according to clause 5 or 6, wherein said first inclined surface portion (34) of said first throughgoing channel (14) and/or wherein said first contact surface (38) of said first valve element (8) is/are having an inclination, relative to the direction D of displacement of said first valve element (8), of 5-90°, such as 10-85°, for example 15-80°, e.g. 20-75°, such as 25-70°, such as 30-65°, for example 35-60°, e.g. 40-55° or 45-50°.

Clause 8. A valve system (100) according to any of the clauses 5-7, wherein a first valve gasket (40) is provided in the area of said first contact surface (38) of said first valve element (8).

Clause 9. A valve system (100) according to clause 8, wherein said first valve gasket (40) is being part of said first inclined surface portion (34) of said first throughgoing channel (14); and/or wherein said first valve gasket (40) is being part of said first contact surface (38) of said first valve element (8).

Clause 10. A valve system (100) according to any of the preceding clauses, wherein said second throughgoing channel (24) of said second valve (4) comprises a widened portion (42) having a second wall segment (44) defining a second inclined surface portion (46) which is being inclined relative to the direction of displacement D of said second valve element (18), and wherein said second valve element (18) comprises a widened portion (48) having a second contact surface (50), wherein said widened portion (48) of said second valve element (18) is being accommodated in said widened portion (42) of said second through-going channel (24) in such a way that when said second valve element (18) is being in its first extreme position, said second contact surface (50) of said second valve element (18) is being in contact with said second inclined surface portion (46) of said second throughgoing channel (24), thereby rendering said second valve (4) closed; and in such a way that when said second valve element (18) is being in its second extreme position, said second contact surface (50) of said second valve element (18) is being separated from said second inclined surface portion (46) of said second throughgoing channel (24), thereby rendering said first valve (4) open.

Clause 11. A valve system (100) according to clause 10, wherein said second contact surface (50) of said second valve element (18) is being inclined relative to the direction of displacement D of said second valve element (18).

Clause 12. A valve system (100) according to clause 10 or 11, wherein said second inclined surface portion (46) of said second throughgoing channel (24) and/or wherein said second contact surface (50) of said second valve element (18) is/are is having an inclination, relative to the direction of displacement of said second valve element (18) of 5-90°, such as 10-85°, for example 15-80°, e.g. 20-75°, such as 25-70°, such as 30-65°, for example 35-60°, e.g. 40-55° or 45-50°.

Clause 13. A valve system (100) according to any of the clauses 10-12, wherein a second valve gasket (52) is provided in the area of said second inclined surface portion (46) of said second throughgoing channel (24).

Clause 14. A valve system (100) according to clause 13, wherein said second valve gasket (52) is being part of said second inclined surface portion (46) of said second throughgoing channel (24); and/or wherein said second valve gasket (52) is being part of said second contact surface (50) of said second valve element (18).

Clause 15. A valve system (100) according to clause 14, wherein said second valve gasket (52) comprises one or more lip portions (54), such as one or more tapered lip portions; wherein said second valve gasket (52) is being part of said second inclined surface portion (46) of said second throughgoing channel (24) and wherein said one or more lip portions (54) is/are pointing towards said second contact surface (50) of said second valve element (18); or wherein said second valve gasket (52) is being part of said second contact surface (50) of said second valve element (18) and wherein said one or more lip portions (54) is/are pointing towards second inclined surface portion (46) of said second throughgoing channel (24).

Clause 16. A valve system (100) according to any of the preceding clauses, wherein the valve body (6) of said first valve (2), at the front end (10) thereof, comprises a depression (56), and wherein the valve body (16) of said second valve (4), at the front end (20) thereof, comprises a hollow protrusion (58) surrounding at least part of said second valve element (18) of said second valve (4), wherein the dimensions and the geometry of said depression (56) and said protrusion (58) are adapted to each other in such a way that said protrusion (58) of said second valve body (16) will fit into said depression (56) of said first valve body (6).

Clause 17. A valve system (100) according to clause 16, wherein said first valve body (6) at the front end (10) thereof and at an inner end of said depression, comprises an end gasket (60), wherein said end gasket (60) surrounds at least part of said first valve element (8) and/or said first through-going channel (14) of said first valve (2), thereby allowing said protrusion (58) of said second valve body (16) of said second valve (4) to abut said end gasket (60), when said protrusion (58) of said second valve body (16) of said second valve (4) in being inserted into said depression (56) of said first valve body (6) of said first valve (2) in order to avoid leaking of gas.

Clause 18. A valve system (100) according to any of the preceding clauses, wherein said first valve element (8) comprises a first part (8a) and a second part (8b), wherein said first part (8a) of said first valve element (8) is being arranged proximate to said front end (10) of said first valve body (6), and wherein said second part (8b) of said first valve element (8) is being arranged distal to said front end (10) of said first valve body (6); wherein said first part (8a) and said second part (8b) of said first valve element (8) are being connected to each other via a threaded tap/threaded hole arrangement (62), thereby allowing adjustment of the total length of said first valve element (8), in a direction parallel to the direction D of displacement of said first valve element (8).

Clause 19. A valve system (100) according to any of the preceding clauses, wherein said first valve element (8), at the end proximate to the front end (10) of said first valve body (6), comprises one or more through-going holes (64) for allowing conveying of gas through said holes into said first throughgoing channel (14) of said first valve body (6).

Clause 20. A valve system (100) according to any of the clauses 8, 9, 13-15 and 17, wherein said gasket (40,52,60) independently is being made of a resilient polymer, such as rubber or silicone.

Clause. A modular incubator system (500) for incubating a viable biological material M, said modular incubator system comprising:
one or more modular incubator chambers (300) in combination with
a docking station (400);
   wherein in respect of one or more of said one or more modular incubator chambers (300), said modular incubator chamber (300) comprises a housing (302) having a first end (340) and a second end (342), thereby defining a longitudinal direction X between said first end and said second end;
   wherein said housing comprises a lid (304), wherein said lid is being configured to be able to shift between an open configuration allowing access to the interior (306) of said modular incubator chamber and a closed configuration, sealing off access to the interior of said modular incubator chamber;
   wherein said modular incubator chamber (300), at said interior (306) thereof, comprises a culture dish support (308) for positioning a culture dish (310) with the view to accommodate one or more biological materials M within the housing (302) of said modular incubator chamber (300);
   wherein in respect of one or more of said one or more modular incubator chambers (300), said housing of said modular incubator chamber (300) comprises a chamber inlet opening for gas (312), wherein said chamber inlet opening for gas (312) is being in fluid connection with the interior (306) of said modular incubator chamber; and
   wherein said housing (302) of said modular incubator chamber furthermore comprises a chamber outlet opening for gas (314), wherein said chamber outlet opening for gas (314) is being in fluid connection with the interior (306) of said modular incubator chamber;
   wherein said docking station (400) comprises one or more docking ports (402) for receiving a modular incubator chamber;
   wherein in respect of one or more docking ports (402) of said docking station (400), said docking port (402) comprises a docking port outlet opening for gas (404); thereby enabling transfer of gas from said docking port (402) of said docking station (400) to the interior (302) of said modular incubator chamber (300) via said docking port outlet opening for gas (404) and said chamber inlet opening for gas (312); and
   wherein said docking port (402) furthermore comprises a docking port inlet opening for gas (406), thereby enabling transfer of gas from the interior (306) of said modular incubator chamber (300) to said docking port (402) of said docking station (400);
   wherein one valve (2,4) of the valve system (100) of any of the clauses 1-20 is being arranged in said chamber inlet opening for gas (312), and wherein another valve (4,2) of the valve system (100) of any of the clauses 1-20 is being arranged in said docking port outlet opening for gas (404); and
   wherein one valve (2,4) of the valve system (100) of any of the clauses 1-20 is being arranged in said chamber outlet opening for gas (314), and wherein another valve (4,2) of the valve system (100) of any of the clauses 1-20 is being arranged in said docking port inlet opening for gas (406).

Clause 22. A modular incubator system (500) according to clause 21, wherein in respect of one or more of said one or more modular incubator chambers (300), and in respect of one or more of said one or more docking ports (402) of said docking station (400), the position of said chamber inlet opening for gas (312) of said modular incubator chamber (300) and the position of said docking port outlet opening for gas (404) of said docking port (402) are adapted to each other in such a way that once docking said modular incubator chamber (300) in said docking port (402), said valve (2,4) of said chamber inlet opening for gas (312) of said housing (302) of said modular incubator chamber (300) and said valve (4,2) of said docking port outlet opening for gas (404) of said docking port (402) will be in fluid connection and in their open configuration; and in such a way that the position of said valve (4,2) of said chamber outlet opening for gas (314) of said modular incubator chamber (300) and the position of said valve (4,2) of said docking port inlet opening for gas (406) of said docking port (402) are adapted to each other in such a way that once docking said modular incubator chamber (300) in said docking port (402), said chamber outlet opening for gas (314) of said housing (302) of said modular incubator chamber (300) and said docking port inlet opening for gas (406) of said docking port (402) will be in fluid connection and in their open configuration.

Clause 23. A modular incubator system (500) according to any of the clauses 21 or 22, wherein in respect of one or more of said one or more modular incubator chambers (300), said housing (302) of said modular incubator chamber (300) comprises a transparent window (316), and wherein in respect of one or more docking ports (402) of said docking station (400), said docking port comprises an image capturing device (408), thereby allowing capturing images of a biological material M being accommodated in the interior (306) of a modular incubator chamber (300), once being docked in said docking port (402).

Clause 24. A modular incubator system (500) according to clause 23, wherein in respect of one or more of said one or more modular incubator chambers (300) and in respect of one or more of said one or more docking ports (402) of said docking station (400), the position of said transparent window (316) of said modular incubator chamber (300) is adapted to the position of said image capturing device (408) in said docking port (402) in a way that enables capturing of images by said image capturing device (408) through said transparent window (316) of said modular incubator chamber (300), once said modular incubator chamber (300) is being docked in said docking port (402).

Clause 25. A modular incubator system (500) according to clause 23 or 24, wherein in respect of one or more of said one or more modular incubator chambers (300), said transparent window (316) of said modular incubator chamber (300) is arranged at a bottom part (357) of said housing (302).

Clause 26. A modular incubator system (500) according to any of the clauses 23-25, wherein in respect of one or more of said one or more modular incubator chambers (300), said transparent window (316) of said housing (302) of said modular incubator chamber is having an elongate shape, such as an elongate and linear extension extending in a direction Y transversal to said longitudinal direction X of said housing of said modular incubation chamber (300).

Clause 27. A modular incubator system (500) according to any of the clauses 23-26, wherein in respect of one or more specific docking ports (402) of said docking station (400), said specific docking port comprises its own dedicated image capturing device (408) which is configured to only capture images relating to a modular incubator chamber (300) which is being docked in said specific docking port (402).

Clause 28. A modular incubator system (500) according to any of the clauses 23-27, wherein in respect of a number N of adjacently arranged docking ports (402) of said docking station (400), said adjacently arranged docking ports share a common image capturing device (408) in the sense that one and only one image capturing device is responsible for capturing images relating to a modular incubator chamber (300) which is being docked in one of said N adjacently arranged docking ports (402), wherein said docking station comprises a displacement device (482), such as an electrically driven and remotely controlled displacement device (482) for enabling displacement of said common image capturing device (408) in relation to said N adjacently arranged docking ports (402) of said docking station (400).

Clause 29. A modular incubator system (500) according to clause 28, wherein said number N is being an integer selected in the ranges of 2-25 or more, such as 4-22, for example 6-20, such as 8-18, such as 10-16 or 12-14.

Clause 30. A modular incubator system (500) according to any of the clauses 21-29, wherein in respect of one or more of said one or more modular incubator chambers (300) and in respect of one or more of said one or more docking ports (402) of said docking station (400), said modular incubator chamber (300) is being configured to be docked in said docking port (402) with its first end (340) facing said docking port (402).

Clause 31. A modular incubator system (500) according to any of the clauses 21-30, wherein in respect of one or more of said one or more modular incubator chambers (300), said modular incubator chamber (300), in the interior 306 thereof, comprises a light source (372) for directing light to the area of the culture dish support (308) of said modular incubator chamber (300), thereby enabling illumination of a viable biological material in a situation of capturing images of said viable biological material.

Clause 32. A modular incubator system (500) according to clause 31, wherein said light source (372) is being attached to said lid (304) of the housing (302) of said modular incubator chamber (300), at an inner side thereof.

Clause 33. A modular incubator system (500) according to clause 31 or 32, wherein said light source (372) is being selected from the group of one or more LEDs, one or more laser diodes, one or more incandescent light bulbs.

Clause 34. A modular incubator system (500) according to any of the clauses 21-33, wherein in respect of one or more of said one or more modular incubator chambers (300), said culture dish support (308) is defining a planar support surface for supporting said culture dish (310).

Clause 35 A modular incubator system (500) according to any of the clauses 21-33, wherein in respect of one or more of said one or more modular incubator chambers (300), said housing (302) of said modular incubator chamber (300), such as at an outer portion thereof, is being provided with electric connectors (322) for providing electric power and/or electric signals to said modular incubator chamber; and wherein in respect of one or more docking ports (402) of said docking station (400), said docking port is being provided with electric connectors (410), thereby allowing providing electric power and/or electric signals from said docking port (402) of said docking station (400) to a modular incubator chamber (300) being docked therein.

Clause 36. A modular incubator system (500) according to any of the clauses 21-35, wherein in respect of one or more of said one or more modular incubator chambers (300), said lid (304) is being a hinged lid which is being connected to said housing of said modular incubator chamber via a hinge.

Clause 37. A modular incubator system (500) according to any of the clauses 21-36 wherein in respect of one or more of said one or more modular incubator chambers (300), said housing (302) of said modular incubator chamber (300) comprises a display (324) which is being configured to display information relating to an operational status of the incubation taking place in said modular incubator chamber.

Clause 38. A modular incubator system (500) according to any of the clauses 21-37, wherein said docking station (400) comprises said docking ports (402) in an arrangement of one or more shelves of adjacently positioned docking ports (402), wherein in case said docking station comprises two or more shelves, said shelves are being arranged above each other.

Clause 39. A modular incubator system (500) according to any of the clauses 21-38, wherein in respect of one or more of said one or more modular incubator chambers (300), said modular incubator chamber comprises an incubation chamber engagement means (326) and wherein in respect of one or more docking ports (402) of said docking station (400), said docking port comprises a docking port engagement means (414), wherein said incubation chamber engagement means (326) is being configured to enter into engagement with said docking port engagement means (414) so as to provide ease and proper positioning and optionally also fixing said modular incubator chamber (300) in said docking port (402), as well as detaching said modular incubator chamber (300) from said docking port (402) of said docking station (400).

Clause 40. A modular incubator system (500) according to any of the clauses 21-39, wherein said modular incubator system (500) comprises an image processing unit (66) 0 for image processing of images captured by said image capturing device(s) (408), wherein said modular incubator system (400) furthermore comprises a data storage (658) for storing images captured by said image capturing units (408) and/or for storing images processed by said image processing unit.

Clause 41. A modular incubator system (500) according to clause 40, wherein one or more of said image capturing devices 408 of said docking ports 402 of said docking station is/are being coupled to said image processing unit 660.

Clause 42. A modular incubator system (500) according to any of the clauses 21-41 wherein in respect of one or more of said modular incubator chambers (300) said valve(s) (2,4) is being arranged with its front end (10,20) pointing outward; and wherein in respect of one or more of said docking ports (402) said valve(s) (4,2) is being arranged with its front end (20,10) pointing outward.

Clause 43. A modular incubator system (500) according to any of the clauses 21-42, wherein in respect of one or more of said one or more modular incubator chambers (300), a first valve (2) of said valve system (100) is being arranged in said chamber inlet opening for gas (312) and in said chamber outlet opening for gas (314); and wherein in respect of one or more of said one or more docking station (402) of said docking station (400), a second valve (4) of said valve system (100) is being arranged in said docking port outlet opening for gas (404) and in said docking port inlet opening for gas (406);
or
wherein in respect of one or more of said one or more modular incubator chambers (300), a second valve (4) of said valve system (100) is being arranged in said chamber inlet opening for gas (312) and in said chamber outlet opening for gas (314); and wherein in respect of one or more of said one or more docking station (402) of said docking station (400), a first valve (2) of said valve system (100) is being arranged in said docking port outlet opening for gas (404) and in said docking port inlet opening for gas (406).

Clause 44. A modular incubator system (500) according to any of the clauses 21-43, wherein said image capturing device (408) comprises microscopic optics so as to enable capturing of microscope images.

Clause 45. A modular incubator system (500) according to any of the clauses 21-44, wherein in respect of one or more of said modular incubator chambers (300), said modular incubator chamber comprises in its interior (306) an electric heating element (318) for heating the interior of said modular incubator chamber, and wherein said modular incubator chamber comprises a power source (320) for providing power to said heating element (318), wherein said electric heating element (318) is being electrically connected to said power source (320).

Clause 46. A modular incubator system (500) according to clause 45, wherein said power source (320) is being an electric power source, such as a battery, for example a rechargeable battery.

Clause 47. A modular incubator system (500) according to any of the clauses 45 or 46, wherein said heating element (318) is being thermally connected to a heat distribution element for distributing heat dissipated in said heating element; wherein said heat distribution element is being arranged, at least partly, in the interior (306) of said modular incubator chamber (300).

Clause. A modular incubator system (500) according to any of the clauses 45-47, wherein said chamber comprises a thermostat (374) and an electric thermostatic circuit (376), wherein said electric heating element (318), said power source (320) and said thermostat (374) are being electrically connected in said electric thermostatic circuit (376) so as to enable thermostatic control of the temperature inside said modular incubator chamber (300).

Clause 49. A modular incubator system (500) according to any of the clauses 21-48, wherein the number of modular incubator chambers (300) of said modular incubator system (500) is selected from the ranges 1-100, such as 2-95, for example 5-90, e.g. 10-85, such as 15-80, for example 20-75, e.g. 25-70, 30-65, such as 35-60, e.g. 40-55 or 45-50.

Clause 50. A modular incubator system (500) according to any of the clauses 21-49, wherein the number of docking ports (402) in said docking station (400) of said modular incubator system 500 is selected from the ranges 1-100, such as 2-95, for example 5-90, e.g. 10-85, such as 15-80, for example 20-75, e.g. 25-70, 30-65, such as 35-60, e.g. 40-55 or 45-50.

Clause 51. A modular incubator system (500) according according to any of the clauses 21-50, wherein in respect of one or more of said docking ports (402) of said docking station (400) of said modular incubator system (500), preferably in respect of all said docking ports (402), said docking port outlet opening for gas (404) comprises a flow restrictor for restricting the magnitude of flow of gas flowing into said docking port (402).

Clause 52. A modular incubator system (500) according to clause 51, wherein said flow restrictor comprises a tube through which the gas is conveyed to said docking port (402), wherein said tube optionally is having a cross-sectional area selected from the ranges of 0.2-8 mm², such as 0.5-7 mm², for example 1-6 mm², such as 2-5 mm² or 3-4 mm²; and/or wherein the length of said tube optionally is selected from the ranges of 5-30 mm, such as 8-25 mm, for example 10-22 mm, e.g. 15-20 mm.

Clause. A modular incubator system (500) according to any of the clauses 21-52, wherein said docking station (400) comprises a gas distribution system (204) for supplying gas to and from one or more of said one or more docking ports (402), wherein said gas distribution system (204) comprises a main gas supply line (210) and a main gas return line (212), wherein in respect of one or more of said docking ports (402), said docking port inlet opening for gas (404) is being fluidly connected to said main gas supply line (210), and said docking port outlet opening for gas (406) is being fluidly connected to said main gas return line (212).

Clause 54. A modular incubator system (500) according to clause 53 wherein said gas distribution system (204) comprises a number of manifold pairs (214), wherein each manifold pair comprises an inlet manifold (216) and an outlet manifold (218), wherein said inlet manifold (216) is being fluidly connected to said main gas supply line (210) and wherein said outlet manifold (218) is being fluidly connected to said main gas return line (212); wherein each manifold pair (214) is connected to one or more docking ports (402) of said docking station (400) in such a way that in respect of a specific manifold pair (214), and in respect of said one or more docking ports (402) being connected thereto, said docking port outlet opening for gas (404) of said docking port (402) is being fluidly connected to said inlet manifold (216), and said docking port inlet opening for gas (406) of said docking port (402) is being fluidly connected to said outlet manifold (218).

Clause 55. A modular incubator system (500) according to clause 53 or 54, wherein said docking station (400) comprises a gas supply system (200), wherein said gas supply system 200 comprises a gas source (202) and said gas distribution system (204), wherein said gas source comprises a supply gas outlet (206) and a return gas inlet (208), wherein said supply gas outlet (206) of said gas source 202 is being fluidly connected to said main gas supply line (210) of said gas distribution system (204), and wherein said return gas inlet (208) of said gas source (202) is being fluidly connected to said main gas return line (212) of said gas distribution system (204).

Clause 56. A modular incubator system (500) according to any of the clauses 53-55, wherein said gas source (202) of said gas supply system (200) comprises a gas mixing box (242) comprising said supply gas outlet (206) and said return gas inlet (208) of said gas source, wherein said main gas supply line (210) of said gas distribution system (204) is being fluidly connected to said supply gas outlet (206), and wherein said main gas return line (212) of said gas distribution system (204) is being fluidly connected to said return gas inlet (208) of said gas source (202), thereby forming a flow loop (244) comprising said gas distribution system (204) and said gas mixing box (242); wherein said flow loop comprises a pump (246) for circulating gas in said loop.

Clause 57. A modular incubator system (500) according to clause 56, wherein said pump (246) is being arranged downstream in relation to said main gas return line (212).

Clause 58. A modular incubator system (500) according to clause 56 or 57, wherein said flow loop (244) comprises a pump oscillation damper (247), wherein said pump oscillation damper optionally is being arranged immediately downstream in relation to said pump (246).

Clause 59. A modular incubator system (500) according to any of the clauses 56-58, wherein said flow loop (244) comprises a pressure sensor, such as a differential pressure sensor (248) for sensing the pressure of gas supplied to said main gas supply line (210) of said gas distribution system (204), wherein said pressure senor (248) optionally is being arranged immediately upstream in relation to said main gas supply line (210) of said gas distribution system (204).

Clause 60. A modular incubator system (500) according to clause 59, wherein said pressure sensor (249) is being a differential pressure sensor, sensing a pressure relative to the pressure of the return gas inlet (208).

Clause 61. A modular incubator system (500) according to any of the clauses 56-60, wherein said flow loop (244) comprises a release valve (249) for enabling pressure relief in said flow loop, wherein said release valve optionally is being arranged immediately downstream in relation to said main gas return line (212) of said gas distribution system (402).

Clause 62. A modular incubator system (500) according to any of the clauses 56-61, wherein said gas mixing box 242 comprises an inlet for N₂ gas (250); and an inlet for CO₂ gas (251), wherein said inlet for N₂ gas (250) is fluidly connected to an N₂ valve (252) for regulating the inflow of N₂, and an N₂ mass flow sensor (253) arranged downstream of said N₂ valve (252) for sensing the amount of N₂ flowing into said gas mixing box (242); and wherein said inlet for CO₂ gas (251) is fluidly connected to a CO₂ valve (254) for regulating the inflow of CO₂, and an CO₂ mass flow sensor (255) arranged downstream of said CO₂ valve (254) for sensing the amount of CO₂ flowing into said gas mixing box (242).

Clause 63. A modular incubator system (500) according to any of the clauses 56-62, wherein said flow loop (244) comprises a mass flow sensor (256) arranged at an upstream position in relation to said gas mixing box (242) for sensing the amount of return gas entering said gas mixing box.

Clause 64. A modular incubator system (500) according to any of the clauses 56-63, wherein said gas source (202) comprises an O₂ sensor 258 for sensing the concentration of O₂ exiting said gas distribution system (204); and wherein said gas source (202) comprises a CO₂ sensor (260) for sensing the concentration of CO₂ exiting said gas distribution system (204), wherein said O₂ sensor and/or said CO₂ sensor optionally is/are being arranged downstream in relation to said pump (246).

Clause 65. A modular incubator system (500) according to any of the clauses 56-64, wherein said gas source (202) comprises a temperature sensor (262) for sensing the temperature of gas circulating in said flow loop (244), wherein said temperature sensor optionally is being arranged downstream in relation to said pump (246), preferably at a position corresponding to the position of said O₂ sensor (258).

Clause 66. A modular incubator system (500) according to any of the clauses 56-65, wherein said gas source (202) comprises a pressure sensor (264) for sensing the absolute pressure in said flow loop (244) wherein said pressure sensor optionally is being arranged downstream in relation to said pump (246), preferably at a position corresponding to the position of said CO₂ sensor (260).

Clause 67. A modular incubator system (500) according to any of the clauses 56-66, wherein said flow loop (244) comprises a UV sanitizer (266) for sanitizing gas flowing in said flow loop (244) via electromagnetic radiation in the UV range, wherein said UV sanitizer optionally being arranged immediately downstream in relation to said main gas return line (212).

Clause 68. A modular incubator system (500) according to any of the clauses 56-67, wherein said gas source (202) comprises one or more filters (268), such as HEPA and/or VOCs filters, wherein such a filter is being arranged immediately upstream in relation to said main gas supply line (210), and/or wherein such a filter is being arranged immediately upstream in relation to the inlet for N₂ gas (250) into said gas mixing box (242); and/or wherein such a filter is being arranged immediately upstream in relation to the inlet for CO₂ gas (251) into said gas mixing box (242).

Clause 69. A modular incubator system (500) according to any of the clauses 56-68, wherein said gas source (202) comprises a gas mixing control system (270), wherein said gas mixing control system is electrically connected to one or more of the following sensors for receiving sensing signals therefrom: said N₂ mass flow sensor (253) for sensing the amount of N₂ flowing into said gas mixing box; said CO₂ mass flow sensor (255) for sensing the amount of CO₂ flowing into said gas mixing box; said mass flow sensor (256) for sensing the amount of return gas entering said gas mixing box; said O₂ sensor (258) for sensing the concentration of O₂ exiting said main gas return line (212) of said gas distribution system (204); said CO₂ sensor (260) for sensing the concentration of CO₂ exiting said main gas return line (212) of said gas distribution system (204); said temperature sensor (262) for sensing the temperature circulating in said flow loop (244); said pressure sensor (264) for sensing an absolute pressure in said flow loop (244), said pressure sensor (248) for sensing the pressure of gas supplied to said gas main gas supply line (210) of said distribution system (204).

Clause 70. A modular incubator system (500) according to clauses 69, wherein said gas mixing control system (270) is electrically connected to one or more of the following elements for control thereof: said N₂ valve (252) for regulating the inflow of N₂ into said gas mixing box (242); said CO₂ valve (254) for regulating the inflow of CO₂ to said gas mixing box (242); said pump (246) for circulating gas in said flow loop (244); said release valve (249).

Clause 71. A modular incubator system (500) according to clause 69 or 70, wherein said gas mixing control system (270) is being configured to receive input from said pressure sensor (248) and on the basis thereof control said pump (246), optionally also to activate said release valve (249) in order to maintain a desired and predetermined pressure of gas supplied to said main gas supply line (210) of said gas distribution system (204).

Clause 72. A modular incubator system (500) according to any of the clauses 69-71, wherein said gas mixing control system (270) is being configured to receive input from said mass flow sensor (256), and on the basis on said input to determine the total amount of CO₂ gas and N₂ gas needed to be supplied via said inlet for CO₂ gas (251) and via said inlet for N₂ gas (250) according to desired and predetermined criteria.

Clause 73. A modular incubator system (500) according to any of the clauses 69-72, wherein said gas mixing control system (270) is being configured to receive input from said CO₂ sensor (260) and said O₂ sensor (258), and on the basis of the CO₂ concentration sensed, is configured to control said CO₂ valve (254), by transmitting a control signal thereto, and thereby regulating the inflow of CO₂ gas in order to reach a desired and predetermined CO₂ concentration, and wherein subsequently, said gas mixing control system (270) on the basis of the O₂ concentration sensed, is configured to control said N₂ valve (252), by transmitting a control signal thereto, and thereby regulating the inflow of N₂ gas in order to reach a desired and predetermined O₂ concentration.

Clause 74. A modular incubator system (500) according to any of the clauses 69-73, wherein said gas mixing control system (270) is configured to use the input from said temperature sensor (262) for compensating the temperature sensitivity of said O₂ sensor (258).

Clause 75. A modular incubator system (500) according to any of the clauses 69-74, wherein said gas mixing control system (270) is configured to use the input from said pressure sensor (264) for compensating the pressure sensitivity of said CO₂ sensor (260).

Clause 76. A modular incubator system (500) according to any of the clauses 69-75, wherein said gas mixing control system (270) is being configured to maintain a pressure of gas supplied to said main gas supply line (210) of said gas distribution system (204), relative to the ambient atmospheric pressure, of 3-20 mbar, such as 5-18 mbar, such as 10-15 mbar above that ambient atmospheric pressure.

Clause 77. A modular incubator system (500) according to any of the clauses 21-76, wherein said modular incubator system comprises a control unit (650) for controlling the operation thereof.

Clause 78. A modular incubator system (500) according to clause 77, wherein said control unit (650) is being coupled to an input device (652), such as an alphanumerical input device for allowing a user to provide settings input relating to a desired operational protocol of said modular incubator system.

Clause 79. A modular incubator system (500) according to clause 77 or 78, wherein said control unit (650) is being coupled to a display unit (654) for displaying, to a user, information relating to settings and/or operational status of said modular incubator system (500).

Clause 80. A modular incubator system (500) according to any of the clauses 77-79, wherein in respect of one or more docking ports (402) of said docking station (400), and or in respect of a modular incubator chamber (300) being docked therein, said control unit (650) is being configured for independently controlling one or more of the following: the setting of said thermostat (374) of a modular incubator chamber (300) being docked therein, switching on and off an active light source (372) of a modular incubator chamber (300) being docked therein and/or regulating the intensity of light emitting from that active light source (372), said gas mixing control system (270); said image capturing unit (408) and/or said associated displacement device (482) of one or more of said docking ports (402) of the docking station (400) of the modular incubator system (500); said image processing unit (660).

Clause 81. A modular incubator system (500) according to any of the clauses 77-80, wherein said control unit (650) is being coupled to a data processing unit (656) and optionally also to a data storage (658) for aiding in handling information during controlling of said modular incubator system.

Clause 82. A modular incubator system (500) according to any of the clauses 77-81, wherein said control unit (650) is being configured for conducting automatic operation of said modular incubator system (500) by independently controlling of one or more of the following: the setting of said thermostat (374) of a modular incubator chamber (300) being docked in a docking port (402), switching on and off an active light source (372) of a modular incubator chamber (300) being docked in a docking port (402) and/or regulating the intensity of light emitting from that active light source (372) of a modular incubator chamber being docked in a docking port (402), said gas mixing control system (270); said image capturing unit (408) and/or said associated displacement device (482) of one or more of said docking ports (402) of the docking station (400) of the modular incubator system (500), said gas mixing control system (270) of said docking station (400) according to predefined control instructions provided thereto, said image processing unit (660).

Clause 83. A modular incubator system (500) according to any of the clauses 77-82, wherein said control unit (650) is being configured for effecting time lapse capturing of images by said image capturing device(s) (408).

Clause 84. A modular incubator chamber (300), wherein said modular incubator chamber (300) comprises a housing (302) having a first end (340) and a second end (342), thereby defining a longitudinal direction X between said first end and said second end;
wherein said housing comprises a lid (304), wherein said lid is being configured to be able to shift between an open configuration allowing access to the interior (306) of said modular incubator chamber and a closed configuration, sealing off access to the interior of said modular incubator chamber;
wherein said modular incubator chamber (300), at said interior (306) thereof, comprises a culture dish support (308) for positioning a culture dish (310) with the view to accommodate one or more biological materials M within the housing (302) of said modular incubator chamber (300);
wherein said housing of said modular incubator chamber (300) comprises a chamber inlet opening for gas (312), wherein said chamber inlet opening for gas (312) is being in fluid connection with the interior (306) of said modular incubator chamber; wherein one valve (2,4) of the valve system (100) according to any of the clauses 1-20 is being arranged in said chamber inlet opening for gas (312);
wherein said housing (302) of said modular incubator chamber furthermore comprises a chamber outlet opening for gas (314), wherein said chamber outlet opening for gas (314) is being in fluid connection with the interior (306) of said modular incubator chamber; wherein one valve (2,4) of the valve system (100) according to any of the clauses 1-20 is being arranged in said chamber outlet opening for gas (314).

Clause 85. A modular incubator chamber (300) according to clause 84, wherein said modular incubator chamber (300) is comprising features as defined in respect of the modular incubator chamber (300) of the modular incubator system (500) according to any of the clauses 21-83.

Clause 86. A docking station (400), wherein said docking station comprises one or more docking ports 402 for receiving a modular incubator chamber (300);
wherein in respect of one or more docking ports (402) of said docking station (400), said docking port (402) comprises a docking port outlet opening for gas (404); thereby enabling transfer of gas from said docking port (402) of said docking station (400) to an interior (302) of said modular incubator chamber (300) via said docking port outlet opening for gas (404); wherein one valve (4,2) of the valve system (100) according to any of the clauses 1-20 is being arranged in said docking port outlet opening for gas (404); and
wherein said docking port (402) furthermore comprises a docking port inlet opening for gas (406), thereby enabling transfer of gas from the interior (306) of a modular incubator chamber (300) to said docking port (402) of said docking station (400); wherein one valve (4,2) of the valve system (100) according to any of the clauses 1-20 is being arranged in said docking port inlet opening for gas (406).

Clause 87. A docking station (400) according to clause 86, wherein said docking station is comprising features as defined in respect of the docking station of the modular incubator system (500) according to any of the clauses 21-83.

Clause 88. Use of a valve system (100) according to any of the clauses 1-20, in a modular incubator system (500).

Clause 89. Use of a modular incubator system (500) according to any of the clauses 21-83, for incubating a viable biological material.

Clause 90. Use of a modular incubator chamber (300) according to any of the clauses 84-85, for incubating a viable biological material.

Clause 91. Use of a docking station (400) according to any of the clauses 86-87, for incubating a viable biological material.

Clause 92. Use according to any of the clauses 88-91, wherein said biological material is being an oocyte or an embryo, such as a human oocyte or a human embryo.

Clause 93. A method of incubating a viable biological material, wherein said method comprises:
i) providing a modular incubator system (500) according to any of the clauses 21-83;
ii) providing a viable biological material;
iii) arranging said viable biological material in a culture dish (310) and subsequently arranging said culture dish in the interior (306) of a modular incubator chamber (300) of said modular incubator system (500);
iv) docking said modular incubator chamber (300) in a docking port (402) of said docking station (400) of said modular incubator system (500);
v) allowing said viable biological material to be incubated in said modular incubator chamber (300);
vi) supplying gas into and out of the interior (306) of said chamber via said valve system (100) of said modular incubator system (500).

Clause 94. A method according to clause 93 further comprising the step of:
viii) removing said incubator chamber (300) from said docking port (402) of said docking station (400), when desired, in order to manually inspect the viable biological material, and optionally also to remove, add or exchange growth medium/media in said culture dish (310).

### LIST OF REFERENCE NUMERALS

2 First valve of valve system
4 Second valve of valve system
6 First valve body of first valve
8 First valve element of first valve
8a First part of first valve element
8b Second part of first valve element
10 Front end of first valve body
12 Rear end of first valve body
14 First throughgoing channel of first valve
16 Second valve body of second valve
18 Second valve element of second valve
20 Front end of second valve
22 Rear end of second valve
24 Second throughgoing channel of second valve
26 First spring of first valve
28 Second spring of second valve
30 Widened portion of first through-going channel
32 First wall segment of widened portion of first through-going channel
34 First inclined surface portion of wall segment of widened portion of first through-going channel
36 Widened portion of first valve element
38 First contact surface of widened portion of first valve element
40 First valve gasket
42 Widened portion of second through-going channel
44 Second wall segment of widened portion of second through-going channel
46 Second inclined surface portion of wall segment of widened portion of second through-going channel
48 Widened portion of second valve element
50 Second contact surface of widened portion of second valve element
52 Second valve gasket
54 Lip portion of second valve gasket
56 Depression of at front end of first valve body
58 Hollow protrusion of second valve body
60 End gasket of first valve
62 Threaded tap/hole arrangement
64 Throughgoing hole in first valve element
100 Valve system
200 Gas supply system
202 Gas source of gas supply system
204 Gas distribution system of gas supply system
206 Supply gas outlet of gas source
20 Return gas inlet of gas source
210 Main gas supply line of gas distribution system
212 Main gas return line of gas distribution system
214 Manifold pair
216 Inlet manifold of manifold pair
218 Outlet manifold of manifold pair
228 Group of docking ports
242 Gas mixing box
244 Flow loop of gas supply system
246 Pump of gas source
247 Pump oscillation damper
248 Pressure sensor for sensing pressure of gas supplied to main gas supply line
249 Release valve
250 Inlet for N₂ gas
251 Inlet for CO₂ gas
252 N₂ valve
253 N₂ mass flow sensor
254 CO₂ valve
255 CO₂ mass flow sensor
256 Mass flow sensor for sensing the amount of return gas flowing into gas mixing box
258 O₂ sensor
260 CO₂ sensor
262 Temperature sensor
264 Pressure sensor
266 UV sanitizer
268 Filter
270 Gas mixing control system
300 Modular incubator chamber
302 Housing of modular incubator chamber
304 Lid of modular incubator chamber
306 Interior of modular incubator chamber
308 Culture dish support
310 Culture dish
312 Modular incubator chamber inlet opening for gas
314 Modular incubator chamber outlet opening for gas
316 Transparent window of housing of modular incubator chamber
318 Electric heating element
320 Electric power source
322 Electric connectors of modular incubator chamber
324 Display of housing of modular incubator chamber
326 Chamber engagement means of modular incubator chamber
340 First end of modular incubator chamber
342 Second end of modular incubator chamber
357 Bottom part of housing of modular incubator chamber
372 Light source
374 Thermostat
376 Thermostatic circuit
400 Docking station
402 Docking port of docking station
404 Docking port outlet opening for gas
406 Docking port inlet opening for gas
408 Image capturing device of docking port of docking station
410 Electric connector of docking port
414 Docking port engagement means of docking port of docking station
482 Displacement device for displacing image capturing unit
500 Modular incubator system
650 Control unit
652 Input device
654 Display unit
656 Data processing unit
658 Data storage
660 Image processing unit
D Direction of displacement of valve element of valve
X Longitudinal direction of modular incubator chamber
Y Transversal direction perpendicular to longitudinal direction

1-93. (canceled)

94. A modular incubator system (500) for incubating a viable biological material M, said modular incubator system comprising:
one or more modular incubator chambers (300) in combination with
a docking station (400);
wherein in respect of one or more of said one or more modular incubator chambers (300), said modular incubator chamber (300) comprises a housing (302) having a first end (340) and a second end (342), thereby defining a longitudinal direction X between said first end and said second end;
wherein said housing comprises a lid (304), wherein said lid is being configured to be able to shift between an open configuration allowing access to the interior (306) of said modular incubator chamber and a closed configuration, sealing off access to the interior of said modular incubator chamber;
wherein said modular incubator chamber (300), at said interior (306) thereof, comprises a culture dish support (308) for positioning a culture dish (310) with the view to accommodate one or more biological materials M within the housing (302) of said modular incubator chamber (300);
wherein in respect of one or more of said one or more modular incubator chambers (300), said housing of said modular incubator chamber (300) comprises a chamber inlet opening for gas (312), wherein said chamber inlet opening for gas (312) is being in fluid connection with the interior (306) of said modular incubator chamber; and
wherein said housing (302) of said modular incubator chamber furthermore comprises a chamber outlet opening for gas (314), wherein said chamber outlet opening for gas (314) is being in fluid connection with the interior (306) of said modular incubator chamber;
wherein said docking station (400) comprises one or more docking ports (402) for receiving a modular incubator chamber;
wherein in respect of one or more docking ports (402) of said docking station (400), said docking port (402) comprises a docking port outlet opening for gas (404); thereby enabling transfer of gas from said docking port (402) of said docking station (400) to the interior (302) of said modular incubator chamber (300) via said docking port outlet opening for gas (404) and said chamber inlet opening for gas (312); and
wherein said docking port (402) furthermore comprises a docking port inlet opening for gas (406), thereby enabling transfer of gas from the interior (306) of said modular incubator chamber (300) to said docking port (402) of said docking station (400);
wherein said modular incubator system (500) comprises a valve system (100), wherein said valve system comprises a first valve (2) and a second valve (4);
wherein said first valve (2) comprises:
a first valve body (6); and
a first valve element (8);
wherein said first valve body (6) comprising a front end (10) and a rear end (12);
wherein said first valve body (6) comprises a first throughgoing channel (14) extending between said front end (10) and said rear end (12) of said first valve body (6);
wherein said first valve element (8) is being arranged in said first throughgoing channel (14) of said first valve body (6) in such a way that said first valve element (8) is being displaceable in a displacement direction D between a first extreme position and a second extreme position in said first throughgoing channel (14), wherein in said first extreme position, said first valve element (8) is being displaced in a direction towards the front end (10) of said first valve body (6), and wherein in said second extreme position, said first valve element (8) is being displaced in a direction towards the rear end (12) of said first valve body (6);
wherein the dimensions and geometries of said first valve body (6) and said first valve element (8) are adapted to each other in such a way that, once being positioned in said first extreme position, said first valve element (8) will block passage through said first throughgoing channel (14) between said front end (10) and said rear end (12) thereof, and in such a way, that once being displaced in a direction towards said second extreme position, said first valve element (8) will provide passage through said first throughgoing channel (14) between said front end (10) and said rear end (12) thereof;
wherein said second valve (4) comprises:
a second valve body (16); and
a second valve element (18);
wherein said second valve body (16) comprising a front end (20) and a rear end (22);
wherein said second valve body (16) comprises a second throughgoing channel (24) extending between said front end (20) and said rear end (22) of said second valve body (16);
wherein said second valve element (18) is being arranged in said second throughgoing channel (24) of said second valve body (16) in such a way that said second valve element (18) is being displaceable in a displacement direction D between a first extreme position and a second extreme position in said second throughgoing channel (24), wherein in said first extreme position, said second valve element (18) is being displaced in a direction towards the front end (20) of said second valve body (16), and wherein in said second extreme position, said second valve element (18) is being displaced in a direction towards the rear end (22) of said second valve body (16);
wherein the dimensions and geometries of said second valve body (16) and said second valve element (18) are adapted to each other in such a way that, once being positioned in said first extreme position, said second valve element (18) will block passage through said second throughgoing channel (24) between said front end (20) and said rear end (22) thereof, and in such a way, that once being displaced in a direction towards said second extreme position, said second valve element (18) will provide passage through said second throughgoing channel (24) between said front end (20) and said rear end (22) thereof;
wherein one valve (2,4) of the valve system (100) is being arranged in said chamber inlet opening for gas (312), and wherein another valve (4,2) of the valve system (100) is being arranged in said docking port outlet opening for gas (404); and
wherein one valve (2,4) of the valve system (100) of is being arranged in said chamber outlet opening for gas (314), and wherein another valve (4,2) of the valve system (100) is being arranged in said docking port inlet opening for gas (406);
wherein the individual incubation chamber is configured in a way that enables support on a planar, horizontal support surface, thereby making the incubator chambers configured to be used for incubation of a viable biological material, irrespective of whether the individual incubator chamber is being docked in a docking port of the docking station, or whether that incubator chamber is removed from the docking port of the docking station.

95. A modular incubator system (500) according to claim 94, wherein the dimensions and geometries of said first valve element (8) and said second valve element (18) are being adapted to each other in such a way that upon bringing said first valve (2) into contact with said second valve (4), by making their respective front ends (10,20) approach each other, said second valve element (18) of said second valve (4) is configured to displace said first valve element (8) of said first valve (2) towards the second extreme position thereof, thereby opening said first valve (2), and further, said first valve element (8) of said first valve (2) is configured to displace said second valve element (18) of said second valve (4) towards the second extreme position thereof, thereby opening said second valve (4).

96. A modular incubator system (500) according to claim 94, wherein said first valve (2) comprises a first spring (26), wherein said first spring is adapted to interact with said first valve element (8), relative to said first valve body (6), in such a way that said first spring (26) will displace said first valve element (8), when not otherwise acted upon, towards said first extreme position thereof, thereby closing said first valve (2);
and/or wherein
said second valve (4) comprises a second spring (28), wherein said second spring is adapted to interact with said second valve element (18), relative to said second valve body (16), in such a way that said second spring (28) will displace said second valve element (18), when not otherwise acted upon, towards said first extreme position thereof, thereby closing said second valve (4).

97. A modular incubator system (500) according to claim 94, wherein in respect of one or more of said one or more modular incubator chambers (300), and in respect of one or more of said one or more docking ports (402) of said docking station (400), the position of said chamber inlet opening for gas (312) of said modular incubator chamber (300) and the position of said docking port outlet opening for gas (404) of said docking port (402) are adapted to each other in such a way that once docking said modular incubator chamber (300) in said docking port (402), said valve (2,4) of said chamber inlet opening for gas (312) of said housing (302) of said modular incubator chamber (300) and said valve (4,2) of said docking port outlet opening for gas (404) of said docking port (402) will be in fluid connection and in their open configuration; and in such a way that the position of said valve (4,2) of said chamber outlet opening for gas (314) of said modular incubator chamber (300) and the position of said valve (4,2) of said docking port inlet opening for gas (406) of said docking port (402) are adapted to each other in such a way that once docking said modular incubator chamber (300) in said docking port (402), said chamber outlet opening for gas (314) of said housing (302) of said modular incubator chamber (300) and said docking port inlet opening for gas (406) of said docking port (402) will be in fluid connection and in their open configuration.

98. A modular incubator system (500) according to claim 94, wherein in respect of one or more of said one or more modular incubator chambers (300), said housing (302) of said modular incubator chamber (300) comprises a transparent window (316), and wherein in respect of one or more docking ports (402) of said docking station (400), said docking port comprises an image capturing device (408), thereby allowing capturing images of a biological material M being accommodated in the interior (306) of a modular incubator chamber (300), once being docked in said docking port (402).

99. A modular incubator system (500) according to claim 98, wherein in respect of one or more of said one or more modular incubator chambers (300) and in respect of one or more of said one or more docking ports (402) of said docking station (400), the position of said transparent window (316) of said modular incubator chamber (300) is adapted to the position of said image capturing device (408) in said docking port (402) in a way that enables capturing of images by said image capturing device (408) through said transparent window (316) of said modular incubator chamber (300), once said modular incubator chamber (300) is being docked in said docking port (402).

100. A modular incubator system (500) according to claim 98, wherein in respect of one or more specific docking ports (402) of said docking station (400), said specific docking port comprises its own dedicated image capturing device (408) which is configured to only capture images relating to a modular incubator chamber (300) which is being docked in said specific docking port (402).

101. A modular incubator system (500) according to claim 98 wherein in respect of a number N of adjacently arranged docking ports (402) of said docking station (400), said adjacently arranged docking ports share a common image capturing device (408) in the sense that one and only one image capturing device is responsible for capturing images relating to a modular incubator chamber (300) which is being docked in one of said N adjacently arranged docking ports (402), wherein said docking station comprises a displacement device (482), such as an electrically driven and remotely controlled displacement device (482) for enabling displacement of said common image capturing device (408) in relation to said N adjacently arranged docking ports (402) of said docking station (400).

102. A modular incubator system (500) according to claim 94, wherein in respect of one or more of said one or more modular incubator chambers (300), said modular incubator chamber (300), in the interior 306 thereof, comprises a light source (372) for directing light to the area of the culture dish support (308) of said modular incubator chamber (300), thereby enabling illumination of a viable biological material in a situation of capturing images of said viable biological material.

103. A modular incubator system (500) according to claim 94, wherein said modular incubator system (500) comprises an image processing unit (66) 0 for image processing of images captured by said image capturing device(s) (408), wherein said modular incubator system (400) furthermore comprises a data storage (658) for storing images captured by said image capturing units (408) and/or for storing images processed by said image processing unit.

104. A modular incubator system (500) according to claim 103, wherein one or more of said image capturing devices 408 of said docking ports 402 of said docking station is/are being coupled to said image processing unit 660.

105. A modular incubator system (500) according to claim 94, wherein in respect of one or more of said one or more modular incubator chambers (300), a first valve (2) of said valve system (100) is being arranged in said chamber inlet opening for gas (312) and in said chamber outlet opening for gas (314); and wherein in respect of one or more of said one or more docking station (402) of said docking station (400), a second valve (4) of said valve system (100) is being arranged in said docking port outlet opening for gas (404) and in said docking port inlet opening for gas (406);
or
wherein in respect of one or more of said one or more modular incubator chambers (300), a second valve (4) of said valve system (100) is being arranged in said chamber inlet opening for gas (312) and in said chamber outlet opening for gas (314); and wherein in respect of one or more of said one or more docking station (402) of said docking station (400), a first valve (2) of said valve system (100) is being arranged in said docking port outlet opening for gas (404) and in said docking port inlet opening for gas (406).

106. A modular incubator system (500) according to claim 94, wherein said docking station (400) comprises a gas distribution system (204) for supplying gas to and from one or more of said one or more docking ports (402), wherein said gas distribution system (204) comprises a main gas supply line (210) and a main gas return line (212), wherein in respect of one or more of said docking ports (402), said docking port inlet opening for gas (404) is being fluidly connected to said main gas supply line (210), and said docking port outlet opening for gas (406) is being fluidly connected to said main gas return line (212).

107. A modular incubator system (500) according to claim 106 wherein said gas distribution system (204) comprises a number of manifold pairs (214), wherein each manifold pair comprises an inlet manifold (216) and an outlet manifold (218), wherein said inlet manifold (216) is being fluidly connected to said main gas supply line (210) and wherein said outlet manifold (218) is being fluidly connected to said main gas return line (212); wherein each manifold pair (214) is connected to one or more docking ports (402) of said docking station (400) in such a way that in respect of a specific manifold pair (214), and in respect of said one or more docking ports (402) being connected thereto, said docking port outlet opening for gas (404) of said docking port (402) is being fluidly connected to said inlet manifold (216), and said docking port inlet opening for gas (406) of said docking port (402) is being fluidly connected to said outlet manifold (218).

108. A modular incubator system (500) according to claim 106, wherein said docking station (400) comprises a gas supply system (200), wherein said gas supply system 200 comprises a gas source (202) and said gas distribution system (204), wherein said gas source comprises a supply gas outlet (206) and a return gas inlet (208), wherein said supply gas outlet (206) of said gas source 202 is being fluidly connected to said main gas supply line (210) of said gas distribution system (204), and wherein said return gas inlet (208) of said gas source (202) is being fluidly connected to said main gas return line (212) of said gas distribution system (204).

109. A modular incubator system (500) according to claim 94, wherein said modular incubator system comprises a control unit (650) for controlling the operation thereof.

110. A modular incubator system (500) according to claim 109, wherein said control unit (650) is being coupled to a data processing unit (656) and optionally also to a data storage (658) for aiding in handling information during controlling of said modular incubator system; optionally wherein said control unit (650) is being configured for effecting time lapse capturing of images by said image capturing device(s) (408).

111. A modular incubator chamber (300), wherein said modular incubator chamber (300) comprises a housing (302) having a first end (340) and a second end (342), thereby defining a longitudinal direction X between said first end and said second end;
wherein said housing comprises a lid (304), wherein said lid is being configured to be able to shift between an open configuration allowing access to the interior (306) of said modular incubator chamber and a closed configuration, sealing off access to the interior of said modular incubator chamber;
wherein said modular incubator chamber (300), at said interior (306) thereof, comprises a culture dish support (308) for positioning a culture dish (310) with the view to accommodate one or more biological materials M within the housing (302) of said modular incubator chamber (300);
wherein said housing of said modular incubator chamber (300) comprises a chamber inlet opening for gas (312), wherein said chamber inlet opening for gas (312) is being in fluid connection with the interior (306) of said modular incubator chamber; wherein one valve (2,4) of the valve system (100) as defined according to claim 1 is being arranged in said chamber inlet opening for gas (312);
wherein said housing (302) of said modular incubator chamber furthermore comprises a chamber outlet opening for gas (314), wherein said chamber outlet opening for gas (314) is being in fluid connection with the interior (306) of said modular incubator chamber; wherein one valve (2,4) of the valve system (100) is being arranged in said chamber outlet opening for gas (314).

112. A docking station (400), wherein said docking station comprises one or more docking ports 402 for receiving a modular incubator chamber (300);
wherein in respect of one or more docking ports (402) of said docking station (400), said docking port (402) comprises a docking port outlet opening for gas (404); thereby enabling transfer of gas from said docking port (402) of said docking station (400) to an interior (302) of said modular incubator chamber (300) via said docking port outlet opening for gas (404); wherein one valve (4,2) of the valve system (100) as defined according to claim 1 is being arranged in said docking port outlet opening for gas (404); and
wherein said docking port (402) furthermore comprises a docking port inlet opening for gas (406), thereby enabling transfer of gas from the interior (306) of a modular incubator chamber (300) to said docking port (402) of said docking station (400); wherein one valve (4,2) of the valve system (100) is being arranged in said docking port inlet opening for gas (406).

113. A method for incubating a viable biological material by using a modular incubator system (500) as defined in claim 94, or by using a modular incubator chamber (300) according to claim 111, or by using a docking station (400) according to claim 112; said biological material optionally is being an oocyte or an embryo, such as a human oocyte or a human embryo.

114. A method of incubating a viable biological material by providing the modular incubator system (500) according to claim 94, the method comprising:
i) providing a viable biological material;
ii) arranging said viable biological material in a culture dish (310) and subsequently arranging said culture dish in the interior (306) of a modular incubator chamber (300) of said modular incubator system (500);
iii) docking said modular incubator chamber (300) in a docking port (402) of said docking station (400) of said modular incubator system (500);
iv) allowing said viable biological material to be incubated in said modular incubator chamber (300);
v) supplying gas into and out of the interior (306) of said chamber via said valve system (100) of said modular incubator system (500).
